(19) United States
(12) Patent Application Publication
(10) Pub. No.: US 2013/0210662 A1
(43) Pub. Date: Aug. 15, 2013

(54) DETECTION OF TARGET METABOLITES

(71) Applicant: Wayne D Frasch, Phoenix, AZ (US)

(72) Inventor: Wayne D Frasch, Phoenix, AZ (US)

(73) Assignee: Arizona Board of Regents,Abody Corporate of the State of Arizona,Acting for andon behalf ofArizona, Scottsdale, AZ (US)

(21) Appl. No.: 13/808,567

(22) PCT Filed: Jul. 7, 2011
(86) PCT No.: PCT/US2011/043160

§ 371 Date: Mar. 18, 2013

Related U.S. Application Data
(60) Provisional application No. 61/362,193, filed on Jul. 7, 2010.

(51) Int. Cl. G01N 33/543

(52) U.S. Cl. CPC G01N 33/54306
USPC 506/9; 435/7.1; 435/287.2; 506/18

(57) ABSTRACT

The present invention provides methods and compositions for highly sensitive detection of a metabolite of interest comprising use of a nanodetection device that comprises an anchoring part, a bridging part and a signal producing part wherein the anchoring part is a molecular motor, the signal producing part is a nanorod and the bridging part is a protein that specifically binds to the metabolite of interest.

### RELATED APPLICATIONS

This application is an International PCT application which claims priority to U.S. Provisional Application No. 61/362,193 filed on Jul. 7, 2010. The entire text of the aforementioned applications is incorporated herein by reference in its entirety.

### BACKGROUND OF THE INVENTION

Rapid and sensitive biosensing of nucleic acids and proteins is vital for the identification of pathogenic agents of biomedical and bioterrorist importance, providing forensic evidence, and for identification of known genotypes using hybrid biological/inorganic devices.

F1-ATPase has recently been used in the construction of a nanoscale rotational device (U.S. Patent Publication No. 2006/0110738) that can be used for single molecule detection (U.S. Pat. No. 6,989,235). These documents demonstrate the feasibility of using a biomolecule to provide a single molecule detection of DNA using a detection signal that is visible by microscopy. The present invention relates to methods of detection that can be used for the detection of targets to a specific protein of interest.

### BRIEF SUMMARY OF THE INVENTION

The present invention provides methods and compositions for highly sensitive detection of a metabolite of interest comprising use of a nanodetection device that comprises an anchoring part, a bridging part and a signal producing part wherein the anchoring part is a molecular motor, the signal producing part is a nanorod and the bridging part is a protein that specifically binds to the metabolite of interest.

In a first embodiments, the invention relates to a nanodetection device comprising an anchoring part, a bridging part and a signal producing part, wherein:
(a) the anchoring part comprises a F1-ATPase molecule modified via site directed mutagenesis so as to comprise a his-tag on the N-terminus of an F1 α or F1-β subunit and a cysteine on the F1-γ subunit wherein a plurality of the F1-ATPase molecule are affixed to the surface of a microscope slide such that each F1-ATPase molecule is oriented with the F1-γ subunit away from the surface of the microscope slide, wherein the cysteine on the F1-γ subunit is biotinylated;
(b) the bridging part comprises a protein that binds to or otherwise responds to the presence of a small molecule metabolite to be detected, wherein the protein in the bridging part is biotinylated and linked to the anchoring part through a biotin-avidin-biotin linkage with the biotinylated F1-γ subunit of the anchoring part; and
(c) the signal producing part comprises an electromagnetic detection probe that has been functionalized with moieties that specifically bind to the protein of the bridging part in the presence of the metabolite to be detected.

More particularly, the protein in the bridging part is a transcriptional regulator protein that comprises a binding site for a specific DNA sequence and a binding site for the small molecule metabolite and the signal producing part comprises specific DNA sequences known to bind the transcriptional regulator.

For example, the transcriptional regulator protein is a transcriptional activator protein wherein assembly of the device occurs upon binding of the specific DNA sequences to the transcriptional activator protein which occurs only in the presence of binding of the small molecule metabolite to the transcriptional regulator protein.

Alternatively, the transcriptional regulator protein is a transcriptional repressor protein wherein assembly of the device occurs upon binding of the specific DNA sequences bound to the transcriptional repressor protein and the electromagnetic detection probe dissociate from the protein in the presence of the small molecule metabolite.

In another alternative, the protein in the bridging part is a signal transduction protein that comprises a binding site for the small molecule metabolite wherein the signal transduction protein changes conformation upon binding to the small molecule metabolite, and the signal producing part comprises an antibody that detects the activated conformation of the signal transduction protein.

In still a further alternative, the protein in the bridging part is a DNA proofing protein that recognizes modified bases in a DNA sequence, and the signal producing part comprises a DNA sequence that is complementary to the DNA sequence to be detected, wherein assembly of the device occurs upon DNA base-pairing of the DNA to be detected with the DNA sequence affixed to the electromagnetic detection probe.

In any of the nanodetection devices described herein the electromagnetic reporter comprises at least one of an optical, magnetic and thermal particle. In particularly preferred embodiments, the electromagnetic reporter is a metal nanorod, preferably a gold nanorod.

In certain embodiments, the electromagnetic reporter comprises an optical reporter comprising at least one of a fluorescent bead and an optical scattering particle.

In the preferred nanodetection devices the electromagnetic reporter is a colloidal particle from the elemental group of metals.

Also contemplated are methods of use. For example, the invention describes a method for detecting whether a molecule binds an activator of a transcriptional regulator protein comprising
(a) preparing a nanodetection device wherein the device comprises an anchoring part, a bridging part and a signal producing part, wherein:
   i) the anchoring part comprises a F1-ATPase molecule modified via site directed mutagenesis so as to comprise a his-tag on the N-terminus of an F1-α or F1-β subunit and a cysteine on the F1-γ subunit wherein a plurality of the F1-ATPase molecule are affixed to the surface of a microscope slide such that each F1-ATPase molecule is oriented with the F1-γ subunit away from the surface of the microscope slide, wherein the cysteine on the F1-γsubunit is biotinylated;
   ii) the bridging part comprises a protein that binds to or otherwise responds to the presence of a small molecule metabolite to be detected, wherein the protein in the bridging part is biotinylated and linked to the anchoring part through a biotin-avidin-biotin linkage with the biotinylated F1-γ subunit of the anchoring part; and
   iii) the signal producing part comprises an electromagnetic detection probe that has been functionalized with moieties that specifically bind to the protein of the bridging part in the presence of the metabolite to be detected,
(b) contacting the device with a target small molecule metabolite;
(c) adding ATP to the device under conditions to allow activity of F1-ATPase to rotate the F1-γ subunit; and
(d) comparing the signal produced from the rotating electromagnetic detection probes bound to the microscope slide in the presence of the target metabolite with the signal produced by probes in the absence of the target metabolite wherein an increase in the signal in the presence of the metabolite indicates that the metabolite is bound to the transcriptional regulator protein.

Also contemplated is a method for detection of a molecule that binds a repressor protein comprising
(a) preparing a nanodetection device wherein the device comprises an anchoring part, a bridging part and a signal producing part, wherein, the protein in the bridging part is a transcriptional regulator protein that comprises a binding site for a specific DNA sequence and a binding site for said small molecule metabolite and said signal producing part comprises specific DNA sequences known to bind said transcriptional regulator,
(b) contacting the device with a target small molecule metabolite;
(c) adding ATP to the device under conditions to allow activity of F1-ATPase to rotate the F1-γ subunit; and
(d) comparing the signal produced from the rotating electromagnetic detection probes bound to the microscope slide in the presence of the target metabolite with the signal produced by probes in the absence of the target metabolite wherein a decrease in the signal in the presence of the metabolite indicates that the metabolite is bound to the repressor protein.

In another embodiment, the invention provides a method of detecting binding of a molecule to a signal transduction protein comprising:
(a) preparing a nanodetection device of the invention in which the protein in the bridging part is a signal transduction protein that comprises a binding site for said small molecule metabolite wherein said signal transduction protein changes conformation upon binding to said small molecule metabolite, and said signal producing part comprises an antibody that detects the activated conformation of said signal transduction protein;
(b) contacting the device with a target small molecule metabolite;
(c) adding ATP to the device under conditions to allow activity of F1-ATPase to rotate the F1-γ subunit; and
(d) comparing the signal produced from the rotating electromagnetic detection probes bound to the microscope slide in the presence of the target metabolite with the signal produced by probes in the absence of the target metabolite wherein an increase in the signal in the presence of the metabolite indicates that the metabolite is bound to the signal transduction protein.

In another embodiments, the invention provides a method of proofing DNA comprising:
(a) preparing a nanodetection device of the invention in which the protein in the bridging part is a DNA proofing protein that recognizes modified bases in a DNA sequence, and said signal producing part comprises a DNA sequence that is complementary to the DNA sequence to be detected, wherein assembly of said device occurs upon DNA base-pairing of said DNA to be detected with the DNA sequence affixed to said electromagnetic detection probe;
(b) contacting the device with a DNA sequence to be proofed;
(c) adding ATP to the device under conditions to allow activity of F1-ATPase to rotate the F1-γ subunit; and
(d) determining the presence of a modified DNA in the DNA sequence to be proofed by determining the signal produced from the rotating electromagnetic detection probes bound to the microscope slide in the presence of the DNA to be proofed wherein a signal is produced when there is DNA base-pairing with a DNA sequence affixed to the electromagnetic detection probe.

In any of the methods of use described herein the step of determining/detecting preferably comprises detecting a signal produced by the signal producing part using visual detection by dark field microscopy. More specifically, the methods comprise determining an oscillation of intensity of light at one or more wavelengths from the detection probe.

Also taught herein are kits for use in identifying the presence of target metabolites, the kits comprising
(a) a protein that specifically binds the small molecule metabolite of interest, wherein the protein is biotinylated in a manner that does not interfere with the binding of the small molecule metabolite of interest;
(b) F1-ATPase molecule wherein the F1-ATPase molecule has a his-tag to facilitate attachment of the F1-ATPase to a solid support and further wherein the F1-ATPase is biotinylated; and
(c) gold nanorods bound to molecules that recognize the protein in (a) when the protein is bound to mall molecule metabolite of interest.

In specific embodiments, the kits may further comprise solid support. In still other embodiments, the kits may further comprise avidin.

The invention also contemplates a method for detecting at least one target small molecule metabolite comprising an anchoring part, a bridging part, and a signal producing part wherein:

(a) the anchoring part comprises a molecular motor, wherein the molecular motor is a biological or synthetic molecule capable of induced translational or rotational movements that are capable of being detected;

(b) the bridging part comprises at least one biological or synthetic component that binds to or otherwise responds to the presence of a small metabolite to be detected, wherein said bridging part is linked to said anchoring part through a covalent or high affinity binding interaction;

(c) the signal producing part that comprises at least one of an electromagnetic detection probe that has been functionalized with moieties that specifically bind to or dissociate from said bridging component in the presence of said metabolite to be detected.

In another embodiments, the invention relates to a method of nanodetection of a target molecule in a sample comprising:

(a) binding each of a plurality of molecular motors to a selected bridging part, wherein said molecular motor is a biological or synthetic molecule capable of detectable translational or rotational movements that are induced and said bridging part is at least one biological or synthetic component that binds to or otherwise responds to the presence of a small metabolite to be detected;

(b) attaching the plurality of molecular motors to a solid support after assembly of said molecular motors with said bridging part;

(c) binding an electromagnetic detection probe specific for the bridging part to the immobilized bridging device wherein said bridging part has a high affinity for said probe in the absence of a target metabolite; and wherein binding of a target metabolite to the bridging part induces a decrease in the affinity of the probe for said bridging part

(d) applying a sample suspected of containing the target metabolite specific for the bridging part;

(e) inducing translational or rotational movement of the at least one molecular motor coupled to the solid support; and

(f) microscopically detecting translational or rotational movement of the at least one molecular motor coupled to the solid support wherein presence of translational or rotational movement in the presence of the sample as indicated by a change in electromagnetic properties of the probe is indicative of lack of target metabolite in the sample and absence of translational or rotational movement as indicated by a lack of change in electromagnetic properties of the probe in the presence of the sample is indicative of presence of the target metabolite in the sample.

Another embodiment of the invention relates to a method of a nanodetection to determine the presence of a target molecule in a sample comprising:

(a) binding each of a plurality of molecular motors to a selected bridging part; wherein said molecular motor is a biological or synthetic molecule capable of detectable translational or rotational movements that are induced and said bridging part is at least one biological or synthetic component that binds to or otherwise responds to the presence of a small metabolite to be detected;

(b) attaching the plurality of molecular motors to a solid support after assembly of said molecular motors with said bridging part;

(c) applying a sample suspected of containing the target metabolite specific to the bridging component in the absence of said electromagnetic probe;

(d) binding of an electromagnetic probe specific for the bridging part wherein said bridging part has a high affinity for the probe when target metabolite specific to said bridging part is bound to said bridging part;

(e) inducing translational or rotational movement of the at least one molecular motor coupled to the solid support; and

(f) microscopically detecting translational or rotational movement of the at least one molecular motor coupled to the solid support by monitoring changes in electromagnetic properties of the probe,

wherein an increased affinity of said probe for the bridging part as indicated by an increase in translational or rotational movement in the presence of the sample indicates the presence of target metabolite specific for that bridging part in the sample, and a decreased affinity of said probe for the bridging part as indicated by a decrease in translational or rotational movement in the presence of the sample indicates the absence of target metabolite specific for that bridging part in the sample.

In yet another embodiments, there is a method of a nanodetection of a target molecule in a sample comprising:
(a) binding each of a plurality of molecular motors to a solid support wherein said molecular motor is a biological or synthetic molecule capable of detectable translational or rotational movements that are induced;
(b) linking to each of the plurality of molecular motors a bridging part that comprises at least one biological or synthetic component that binds to or otherwise responds to the presence of a small metabolite to be detected;
(c) binding an electromagnetic detection probe specific for the bridging part to the immobilized bridging device wherein said bridging part has a high affinity for said probe in the absence of a target metabolite, and wherein binding of a target metabolite to the bridging part induces a decrease in the binding affinity of the probe for said bridging part;
(d) applying a sample suspected of containing the target molecule or metabolite specific for the bridging part;
(e) inducing translational or rotational movement of the at least one molecular motor coupled to the solid support; and
(f) microscopically detecting translational or rotational movement of the at least one molecular motor coupled to the solid support wherein presence of translational or rotational movement in the presence of the sample as indicated by a change in electromagnetic properties of the probe is indicative of lack of target metabolite in the sample and absence of translational or rotational movement as indicated by lack of change of electromagnetic properties of the probe is indicative of presence of metabolite in the sample.

In still a further embodiments of the invention there is a method of a nanodetection to determine the presence of a target molecule in a sample comprising:
(a) binding each of a plurality of molecular motors to a solid support wherein said molecular motor is a biological or synthetic molecule capable of detectable translational or rotational movements that are induced;
(b) linking to each of the plurality of molecular motors a bridging part that comprises at least one biological or synthetic component that binds to or otherwise responds to the presence of a small metabolite to be detected;
(c) applying a sample suspected of containing the target metabolite specific to the bridging component in the absence of said electromagnetic probe;
(d) binding of an electromagnetic probe specific for the bridging part wherein said bridging part has a high affinity for the probe when target molecule specific to said bridging part is bound to said bridging part;
(e) inducing translational or rotational movement of the at least one molecular motor coupled to the solid support; and
(f) microscopically detecting translational or rotational movement of the at least one molecular motor coupled to the solid support by monitoring changes in electromagnetic properties of the probe,

wherein an increased affinity of said probe for the bridging part as indicated by an increase in translational or rotational movement in the presence of the sample indicates the presence of target metabolite specific for that bridging part in the sample, and a decreased affinity of said probe for the bridging part as indicated by a decrease in translational or rotational movement in the presence of the sample indicates the absence of target metabolite specific for that bridging part in the sample.

In the above methods, it should be understood that step (c) may be performed prior to, after or concurrently with step (d).

In any of the methods of the invention the bridging part is a transcriptional regulator protein that comprises a binding site for a specific DNA sequence and a binding site for said small molecule metabolite and said signal producing part comprises specific DNA sequences known to bind said transcriptional regulator. Preferably, the transcriptional regulator protein is a transcriptional activator protein wherein assembly of said device occurs upon binding of said specific DNA sequences to said transcriptional activator protein which occurs only in the presence of binding of said small molecule metabolite to said transcriptional regulator protein. Alternatively, the transcriptional regulator protein is a transcriptional repressor protein wherein assembly of said device occurs upon binding of said specific DNA sequences bound to said transcriptional repressor protein and said electromagnetic detection probe dissociate from said protein in the presence of said small molecule metabolite.

In some embodiments, the bridging part is a signal transduction protein that comprises a binding site for said small molecule metabolite wherein said signal transduction protein changes conformation upon binding to said small molecule metabolite, and said signal producing part comprises an antibody that detects the activated conformation of said signal transduction protein.

The bridging part may be a DNA proofing protein that recognizes modified bases in a DNA sequence, and said signal producing part comprises a DNA sequence that is complementary to the DNA sequence to be detected, wherein assembly of said device occurs upon DNA base-pairing of said DNA to be detected with the DNA sequence affixed to said electromagnetic detection probe.

In the present invention the electromagnetic reporter or probe comprises at least one of an optical, magnetic and thermal particle. For example, the electromagnetic reporter or probe comprises an optical reporter comprising at least one of a fluorescent bead and an optical scattering particle. In exemplary embodiments, the electromagnetic reporter or probe is a colloidal particle from the elemental group of metals.

### BRIEF DESCRIPTION OF SEVERAL VIEWS OF THE DRAWINGS

FIG. 1: Dark Field Microscopy set up for nanodetection of metabolites.

FIG. 2A through 2C: Micrograph of light scattered from a single gold nanorod viewed through a polarizing filter when nanorod is positioned at 0°, 45°, and 90° to the polarizing filter. The diagram shows attachment of the gold nanorod to the rotating arm (gamma subunit) of the F1-ATPase. In position A (0°) the color is green, in position C, the color is red. In position B, the color is not orange but rather a mixture of intensities of green and red scattered light from the nanorod.

FIG. 3: Bar graph depicting the detection of Stx2 toxin protein in cell supernatant based on the number of nanorods observed in 30 consecutive fields using cell supernatants containing E. coli EDL933 Stx2 producing strain (FIG. 3A, 3C), or E. coli MG1655 non-toxin producing strain (FIG. 3B, 3D). Nanodevices in FIG. 3C and FIG. 3D were controls that were missing the capture (anti-Stx2B) antibody.

FIG. 4: Bar graph depicting the detection of Stx2 toxin protein in a sample containing purified toxin or from a saliva sample containing either EDL933 (toxin producing strain) or MG1655 (non-toxin producing strain).

FIG. 5: Bar graph depicting the detection limit of Stx2 toxin protein in cell supernatants measured in 10 consecutive fields of view by nanodevice assembly (blue) versus the number of rotating nanorods in 10 (red bars) or 30 (green bars) consecutive fields of view.

FIG. 6: Bar graphs depicting rotation-based detection limit of purified Stx2 toxin protein determined in 10 consecutive field of view, with the data expressed as a function of the number of target molecules (FIG. 6A) or the target concentration (FIG. 6B) in the samples.

FIG. 7: Summary of criteria used in algorithms employed to identify and quantify the number of red, green, blue and yellow nanorods in a digital photo of a field of view.

FIG. 8: Depiction of self-assembling nanodevices for multiplexed detection of the Stx2 toxin protein (left, red nanorods) and the stx1 gene DNA sequence (right, green nanorods) in the same sample well.

FIG. 9: Bar graphs depicting the simultaneous detection of Stx2 protein and the stx1 gene DNA sequence in the same sample using red and green nanorods, respectively in a well containing supernatant from E. coli strain EDL933 (+toxin) or Mg1655 (−toxin).

FIG. 10: Depiction of metabolite detection using a transcriptional activator protein. FIG. 10A, the target metabolite (cAMP) binds to the activator protein (CAP) that is immobilized on the rotating arm of the F₁ molecular motor. FIG. 10B depicts the binding of cAMP to CAP induces conformational change that creates a high affinity binding site for the DNA receptor sequence. FIG. 10C depicts gold nanorods coated with the specific DNA receptor sequences bind to the CAP-cAMP complex to complex nanodevice assembly and enable metabolite detection by microscopy.

FIG. 11: Crystal structure of the assembled components (left, including F₁, avidin, CAP and DNA) and depiction of nanodevice components using a transcriptional regulatory protein as an actuator for metabolite detection.

FIG. 12: Depiction of the metabolite detection using a transcriptional repressor protein. FIG. 12A depicts a target metabolite (NADH) binds to the repressor protein (NRP) that is immobilized on the rotating arm of the F₁ molecular motor. FIG. 12B depicts the binding of NADH to NRP displaces NAD+ and induces a conformational change that eliminates the binding site for the DNA receptor sequence. FIG. 12C depicts the gold nanorods coated with the specific DNA receptor sequences are removed by a wash and the metabolite is detected by a decrease in the number of bound nanorods on the microscope slide.

FIG. 13: Depiction of ordered nanodevice arrays for metabolite detection using NADH.

FIG. 14: Depiction of the use of E-beam lithography to construct nanoscale nickel islands on a microscope slide in a pattern with controlled dimensions.

### DETAILED DESCRIPTION OF THE INVENTION

In the present invention, there is provided a method for the detection of target metabolites using a nanodetection method that employs immobilized F₁-ATPase. A wide variety of transcriptional regulator proteins exist, each of which binds a small molecule with high affinity and specificity. Such transcriptional regulator proteins are responsible for activating or repressing DNA transcription as a result of binding to specific DNA sequences. Most of these regulator proteins only bind to DNA in dimeric form, which occurs as the result of the occupancy by the signal molecule. For transcriptional activating proteins, metabolite binding induces dimerization and formation of the high affinity binding site to bind DNA. In repressor proteins, the binding of metabolite has the opposite effect. Examples of regulator proteins include but are not limited to the catabolite activator protein that binds cyclic AMP, arabinose transcriptional activator that binds arabinose, uracil-dependent transcriptional regulator, GTP and isoleucine responsive regulator, trypophan repressor protein, and maltose transcriptional activator, glucose-resistance amylase regulator.

The target metabolites include but are not limited to amino acids, carbohydrates, nucleotides, nucleosides, hormones, organic acids, and any small molecule that will bind specifically to a transcriptional regulator protein. Regulator proteins can be genetically modified to change the specificity of the metabolite. In one embodiment, the arabinose transcriptional activator may be mutated so that it becomes specific for the binding of glucose in lieu of arabinose.

In the present invention, there are provided nanodetection devices that can be used to detect specific metabolites. Exemplary such devices are constructed by mounting a molecule of the F₁-ATPase on the surface of a microscope slide oriented with the rotary γ-subunit axel away from the surface. This will be achieved by the presence of his-tags on the C-termini of the α and/or the β subunits of F₁, which has high affinity binding to the surface of a microscope slide that is unmodified or has been functionalized to contain nickel-NTA. The F₁-ATPase γ subunit is genetically modified to contain a cysteine that will be covalently modified to contain a biotin moiety via, for example, biotin-maleimide.

The transcriptional regulator protein that will provide the specificity for detection of the target molecule (metabolite) will be genetically modified to contain a cysteine for biotinylation that is located so as not to interfere with dimerization or with the DNA binding site. The biotinylated regulator protein and biotinylated F₁-ATPase are then assembled using avidin.

The surface of a detection probe that is visible under a microscope, including but not limited to gold nanorods, will be functionalized to contain double-stranded DNA with the sequence specific to the regulator protein. Detection will be made when the target molecule (metabolite) binds to the regulator protein, which induces dimerization and formation of the high affinity DNA binding site. Subsequent addition of the functionalized nanorods will allow complex assembly of the nanoscale detection device only for those regulator proteins that have bound the metabolite. Detection can be made by comparing the number of nanorods bound on the microscope slide in the presence versus the absence of the target metabolite. The detection limit using the assembly of nanodevices is the point at which the number of target-specific assembly of the nanodevices is not significantly increased above the number of gold nanorods that are bound nonspecifically to the surface of the microscope slide. Under these limiting conditions, addition of ATP provides fuel to the F₁-ATPase molecular motor to induce rotation. The only nanorods that rotate are those that are bound specifically to the correctly assembled nanodevices that contain the target metabolite.

Detection of ATP can be made by attaching the gold Nanorod directly to the F₁-ATPase γ subunit, since the presence of ATP will induce rotation. In a similar manner the presence of ADP can be made using a coupled enzyme assay with a high concentration of pyruvate kinase and phosphoenol pyruvate. Likewise, the glucose-resistance amylase regulator assembled with F₁-ATPase can be used to detect glucose via an enzyme coupled assay with hexokinase and ATP to convert glucose to glucose-6-phosphate (G-6-P). The G-6-P then binds to the regulator protein to facilitate assembly with the DNA-functionalized gold Nanorod for detection.

Detection using a repressor protein is accomplished by assembling the repressor protein dimer with the F₁-ATPase and the DNA-functionalized Nanorod. In this case the repressor protein dimer has a high affinity for the DNA in the absence of the target metabolite. Binding of the target metabolite to the repressor protein causes the dissociation of the complex, which is detected as a decrease in the number of nanorods observed bound to the surface of the microscope slide. To facilitate quantification of the number of target metabolites that bind, the nanodevices can be assembled at specific intervals on the microscope slide surface in a nanoarray pattern. This will give rise to a grid pattern of the nanorods that will be disrupted upon binding of target metabolite. Quantification is determined by the number of nanorods observed before and after addition of the target metabolite.

In another embodiment, the device is constructed using signal transduction protein kinases to detect target metabolites. Several protein kinases are activated by the binding of specific metabolites. A specific embodiment is protein kinase A, in which the kinase catalytic activity becomes activated as the result of binding cyclicAMP. The protein contains two catalytic subunits and two regulatory subunits that bind cAMP. Binding of cAMP to the regulatory subunits induces a conformational change that causes the dissociation and activation of the catalytic subunits. To detect a target metabolite using one of these kinases, the regulatory subunits of the kinase will be attached to the biotinylated F₁-ATPase via a genetically modified cysteine that has been biotinylated using biotin-avidin links. This assembly will be designed so that the catalytic subunits are distal from the F₁-ATPase. Upon binding of the target metabolite, the catalytic subunits will dissociate, thereby exposing a previously sequestered region of the regulatory subunits. Addition of gold nanorods that have been functionalized with a monoclonal antibody to that part of the regulatory subunit that is exposed upon activation will allow assembly of the nanodevice, and thereby enable detection.

In another embodiment, the devices are constructed such that the protein component forming the bridge between the nanorod/nanoparticle and the F1-ATPase are DNA proofreading proteins that are used for detection of modified DNA. Cytosine methylation in DNA is a major epigenetic signal that plays a central role in propagating chromatin status during cell division. Proofreading proteins bind specifically to sequences of DNA that contain modified bases like 5-methyl cytosine. For example, the ubiquitin-like, containing PHD and Ring Finger Domains (UHRF1) SRA domain is required for CpG maintenance methylation at DNA replication forks. This protein binds specifically to 5-methyl cytosine in DNA sequences and flips the base out of the DNA helix.

The proofreading protein responsible for binding to a specific modification of DNA will attached via an avidin-biotin link to the F₁-ATPase. The single stranded target DNA strand that contains the modified base will be introduced to this detection complex, or will be hybridized to the complementary DNA strand known as the detection probe strand. This complementary strand will be modified to contain biotin covalently at either end for attachment to Avidin coated gold nanorods for assembly of the nanodevice that enables detection.

The present invention thus provides novel devices and methods for using such devices for extremely sensitive detection of target metabolites in a given sample. The methods disclosed herein detect a target small molecule metabolite as the result of the interaction of that small molecule metabolite to be detected with a binding protein that specifically binds that small molecule metabolite and produces a signal in the device only in the presence of the specific small molecule metabolite to be detected. A specific protein which binds to the small molecule metabolite of interest is used to bridge a molecular motor that is anchored to a solid surface and a detection probe. However, the bridge is only formed when the protein is bound to the small molecule metabolite to be detected. When this binding occurs, it is revealed through the detection probe which reveals the motion imparted by the molecular motor to the bridging part that is indicative of the binding of the small molecule metabolite to be detected to the protein in the bridging part. The motion imparted to the probe is observed by an appropriately chosen means of detection of signal from the detection probe. The methods of the invention are capable of detecting single molecules of the small molecule metabolite to be detected, and thus provide an extremely sensitive technique for target detection that is of wide applicability, including but not limited to clinical diagnostics, forensic analysis, gene expression analysis, DNA sequencing and proofing, and DNA computing.

The devices used in the present invention can be described as being comprised of three separate parts: an anchoring part, a bridging part and a signal producing part. The anchoring part will be that part that anchors the entire assembly to a microscope slide and allows the microscope-based detection of the signal produced. The anchoring part comprises F₁-ATPase molecule modified via site directed mutagenesis so as to comprise a his-tag on the N-terminus of an F1-α or F1-β subunit and a cysteine on the F1-γ subunit wherein a plurality of said F1-ATPase molecule are affixed to the surface of a microscope slide such that each F1-ATPase molecule is oriented with the F1-γ subunit away from the surface of said microscope slide, wherein said cysteine on the F1-γ subunit is biotinylated. The bridging part comprises a protein that binds to or otherwise responds to the presence of a metabolite to be detected, wherein said protein in said bridging part is biotinylated and linked to said anchoring part through a biotin-avidin-biotin linkage with said biotinylated F1-γ subunit of said anchoring part; and the signal producing part comprises an electromagnetic detection probe that has been functionalized with moieties that specifically bind to said protein of said bridging part in the presence of said metabolite to be detected.

The small molecule metabolite to be detected can be any molecule that can be bind to a protein of interest where the protein is used as the protein in the bridge between a molecular motor and a detection probe to detect motor-induced motion and for which the means of formation of that bridge is specific to that small molecule metabolite to be detected. The small molecule metabolite to be detected can be any molecule that can be bind to a protein of interest where the protein is used as the protein in the bridge between a molecular motor and a detection probe to detect motor-induced motion and for which the means of formation of that bridge is specific to that small molecule metabolite to be detected.

The sample in which detection of small molecule metabolite to be detected is to be performed can be any sample of interest, including but not limited to synthetic nucleic acids, genomic DNA, cell lysates, tissue homogenates, forensic samples, environmental samples, and isolated nucleic acid samples from cells, tissues, or complete organisms. In addition, the sample may be from a library of small molecules that can be used to identify agents that bind to the protein that is part of the bridging portion. In this manner new modulators of the protein that forms the bridging portion of the device may be identified.

Optimization of conditions for contacting a sample containing or suspected of containing the small molecule metabolite to be detected to nanodetection device under conditions whereby the small molecule metabolite to be detected will bind to the protein in the bridging portion of the device can be readily accomplished by those of skill in the art.

### A. THE ANCHORING PART

The anchoring part of the nanodetection device is comprised of a molecular motor that is any biological or synthetic molecule capable of induced translational or rotational movements that are capable of detection. In a preferred embodiment, the molecular motor comprises a biomolecular motor. Non-limiting examples of such biomolecular motors comprise F1-ATPases, actomyosin, ciliary axonemes, bacteria flagellar motors, kinesin/microtubules, and nucleic acid helicases and polymerases.

In a preferred embodiment, the molecular motor comprises an F1-ATPase. The F1-ATPase enzyme includes alpha-subunits and beta-subunits in its base structure. Base structure is non-rotating and anchored or attached preferentially to surface. Surface can be part of a DNA microarray, glass slide, or other dielectric substrate.

By way of example, the rotating subunits on the F1 ATPase include the γ and ∈ subunits, while the α, β, and δ subunits do not rotate. Thus, where the F1 ATPase is used as the molecular motor, it is preferred that the first affinity tag bind (directly or indirectly) to the y and/or s subunits, while the molecular motor is attached via a functional moiety, such as a his-tag, to a substrate through the α, β, or δ subunits. In a preferred embodiment, the F1-ATPase rotary biomolecular motor is a complex of α3β3γ subunits. This complex provides optimal binding of the F1 ATPase to the solid support and the γ subunit to the DNA. Details of F1-ATPase assembly, subunit composition, and inducement of F1-ATPase rotation are well known to those of skill in the art; see, for example, US 20030215844; Yoshida et al., Journal of Biological Chemistry, 252: 3480-3485 (1977); Du et al., Journal of Biological Chemistry, 276: 11517-11523 (2001); Bald et al., Journal of Biological Chemistry, 275: 12757-12762 (2000); Kato-Yamada et al., Journal of Biological Chemistry, 273: 19375-19377 (1998), Kato et al., Journal of Biological Chemistry, 272: 24906-24912 (1997); Tucker et al., Journal of Biological Chemistry, 279: 47415-8 (2004); Tucker et al., Eur. J. Biochem. 268: 2179-86 (2001), and Du et al., Journal of Biological Chemistry, 276: 11517-23 (2001).

Exemplary non-limiting examples of F1-ATPase subunits that can be used in the methods and compositions of the present invention are known to those of skill in the art and include those that are deposited at Genbank Accession number NM_{—}128864; Genbank Accession number NM 121348; Genbank Accession number NM 104043; Genbank Accession number D14699; Genbank Accession number D14700; AB095026; Genbank Accession number BT000409; Genbank Accession number AY136289; Genbank Accession number AY114540; Genbank Accession number AJ487471; Genbank Accession number M20929; Genbank Accession number AF034118; Genbank Accession number D10491; Genbank Accession number X05366; Genbank Accession number AY072309; Genbank Accession number AY062627; Genbank Accession number U61392; Genbank Accession number U61391; Genbank Accession number X05970; Genbank Accession number AB044942; Genbank Accession number AF052955; Genbank Accession number D37948; Genbank Accession number AB022018; Genbank Accession number AB007034; Genbank Accession number D15065; Genbank Accession number D00022; Genbank Accession number J05397; AF134892; Genbank Accession number Z00018; Genbank Accession number U46215; Genbank Accession number X53537; Genbank Accession number X03559; Genbank Accession number AF010323; Genbank Accession number AB003549; Genbank Accession number D88377; Genbank Accession number D88376; Genbank Accession number D88375; Genbank Accession number D88374; Genbank Accession number D10660; Genbank Accession number X68691; Genbank Accession number X56008; Genbank Accession number X55389; Genbank Accession number X59066; Genbank Accession number L13320; Genbank Accession number X51422; Genbank Accession number Z00026; Genbank Accession number X07745; X55963; Genbank Accession number X68690; Genbank Accession number X56133; Genbank Accession number V00312; Genbank Accession number U37764; Genbank Accession number M65129; Genbank Accession number J03218; Genbank Accession number M16222; Genbank Accession number J02603; and Genbank Accession number U09305.

The F1-ATPase is preferably immobilized for rotation visualization techniques or if the detection depends on the perturbation of the local environment, such as micro current or impendence. A series of molecular motors, either identical or two or more different molecular motors, can be immobilized on a surface to generate an array of nanodetection devices of the invention. If each F1-ATPase is attached to a different protein in the bridging part of the device, and the different devices are labeled with different labels, this molecular motor array can be used to detect multiple small molecule metabolites in a manner similar to use of a gene chip. As used herein, an “array” comprises a solid surface, with molecular motors attached to said surface. In some embodiments, the array is a random array of a plurality of nanodetection of the invention attached to the solid surface where the devices detect the same or different small molecule metabolites (i.e., the proteins in the bridging part of the nanodetection device detect different small molecules). In other embodiments, the array is an “addressable” array in which the location of specific nanodetection device containing a specific protein is given a specific position on the array and the position is denoted by a difference in color or other selected marker that allows the location of the specific molecular motor to be pinpointed.

Arrays typically comprise a plurality of molecular motor linked to different capture groups that are coupled to a surface of a substrate in different, known locations. For example, there are several silane derivatives to attach a variety of functional groups to a glass surface. The term “solid surface” as used herein refers to a material having a rigid or semi-rigid surface.

Such materials will preferably take the form of chips, plates, slides, cover slips, small beads, pellets, disks or other convenient forms, although other forms may be used. The surfaces are generally coated with an affinity target. Such solid surfaces can be coated in any way that improves desired binding to its surface and/or minimizes non-specific binding to its surface. In a preferred embodiment, nickel-nitrilotriacetic acid (Ni-NTA) affinity resin (Sigma-Aldrich product # P6611) is used. In a further embodiment, acetylated BSA can be added to reduce non-specific binding. Arrays can be fabricated, for example, using electron beam lithography.

Gamma-subunit of the F1-ATPase enzyme is molecularly coupled to base structure and oriented upward, normal to surface of the substrate. The F1-ATPase enzyme operates as a molecular motor with the alpha-subunits and beta-subunits inducing rotation of the gamma-subunit arm. Thus, the gamma-subunit arm behaves as a drive shaft, oriented upward, perpendicular to surface to which the enzyme is attached, and rotating in response to activity in alpha-subunits and beta-subunits. The rotation of gamma-subunit arm is difficult to directly see under a microscope and hence the gamma subunit is coupled to a signal producing part.

### B. THE SIGNAL PRODUCING PART AND DETECTION OF THE SIGNAL

In the signal producing part of the nanodetection device of the invention there is a detection probe. The detection probe can be anything that is capable of attaching to the bridging part of the device and providing a means of detecting the movement generated by the molecular motor, such as metallic nanoparticles (rods, spheres, quantum dots, etc.) fluorescent dyes, and nanoparticles labeled with fluorescent dyes. In a preferred embodiment, elemental metal nanorods are used, including but not limited to gold, silver, aluminum, platinum, copper, zinc, and nickel. In one example, gold rod detection probes capable of visual observation by microscope are functionalized to have attached thereon via a biotin-avidin bond a moiety that binds to the bridging part protein when that protein is bound to a small molecule to be detected.

A gold nanorod is attached to gamma-subunit arm with a protein or other bonding molecule, such as avidin. Gamma-subunit arm can attach to any part of gold nanorod, e.g., at either end, in the middle, or any point in-between. The bonding molecule links gold nanorod to gamma-subunit arm so that the rotational motion of gamma-subunit arm is imparted to gold nanorod. Gold nanorod spins around with the rotational motion of gamma-subunit arm. In a further example, the gold nanorod is coated with anti-DIG antibody (the affinity target), which binds specifically to a DIG (Digoxigenin) second affinity tag.

Metal nanoparticles, such as gold nanorod, are efficient absorbers and scatterers of light owing to collective oscillations of their conduction electrons known as surface plasmons. The number, position, and shape of the surface plasmon bands are determined by the kind of metal, the size and shape of the particle, and the dielectric constant of the surrounding medium. Non-spherical nanoparticles possess multiple surface plasmon modes. For example, rod-shaped nanoparticles exhibit two resonant modes, corresponding to their long and short axes, respectively.

Gold nanorod is shaped as a rod, shaft, or cylinder with rounded or flat ends. Gold nanorod is about 15-40 nm in diameter and about 60-80 nm in length along is symmetrical axis. In other embodiments, gold nanorod has a length-diameter aspect ratio between 2.5:1 and 20:1. The rod-shaped nanoparticle is typically grown from a base wafer or sphere. Gold nanorod exhibits optical anisotropy in that it has two surface plasmon resonances that correspond to the diameter and length of the shaft. The short axis corresponds to the transverse plasmon resonance of the rod. The long axis corresponds to the longitudinal plasmon resonance of the rod. The short and long axes of gold nanorod scatter incident white light at different wavelengths as a function of the respective dimensions. The longer axis has greater surface area than the shorter axis. The short axis or end surface of gold nanorod scatters light at a shorter wavelength due to the lesser surface area and the long axis or side surface of gold nanorod scatters light at a longer wavelength due to its larger surface area. In one embodiment, the short axis of gold nanorod scatters green light having a wavelength of about 520-570 nm, while the long axis scatters red light having a wavelength of 685-730 nm. Other relative dimensions of gold nanorod will scatter incident white light at two different wavelengths.

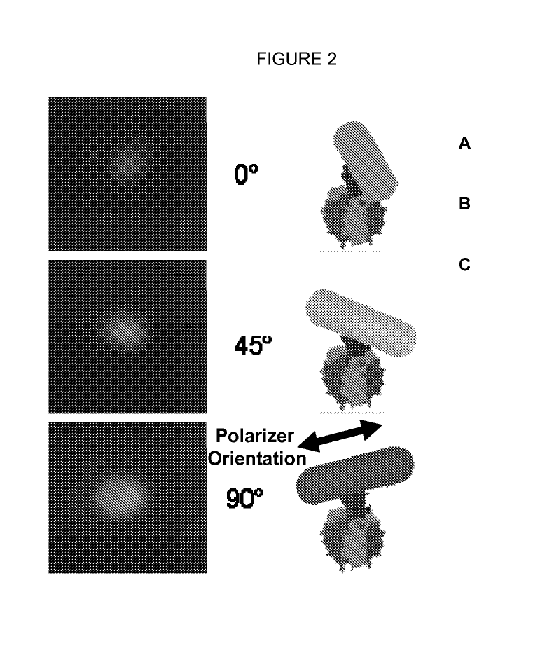

The optical scattering characteristics of gold nanorod, in combination with its rotational motion imparted by gamma-subunit arm, allows for observation and measurement of physical characteristics and behavioral phenomena of molecular structures on the nanoscale. In a simplified view, a full spectrum, white-light source is generally directed in reference direction. The white light photons strike gold nanorod as a wave function and, depending on the orientation of gold nanorod with respect to the incident angle of a polarizing filter, some wavelengths of the light will be scattered. With the given dimensions of gold nanorod, when the nanorod is aligned so that the short axis is parallel to the polarizer (position A), scattering of green light maximal and that of red light is minimal. When the long axis of the nanorod is aligned with the direction of polarization (position B), scattering of red light is maximal and green light minimal. This can be seen in FIG. 2.

The scattered light from gold nanorod is polarized. The intensities of the red and green resonances depend on the relative orientation of the nanorod to the plane of polarization of the incident light. The rotating nature of gold nanorod, due to its connection to the spinning F1-ATPase molecular motor, results in an observable light that flashes or blinks alternating green and red. The intensity and rate of blinking of the red and green light is a function of the speed of rotation of gamma-subunit arm. By observing the red and green blinking light from gold nanorod, the rotational motion of F1-ATPase enzyme can be detected and measured. The visible blinking light is much easier to observe than the physical rotating structure itself. Moreover, the polarized nature of the scattered wavelength causes gold nanorod to go dark in between positions A-D. The flashing green and red lights are detectable, observable, and measurable using dark field microscopy.

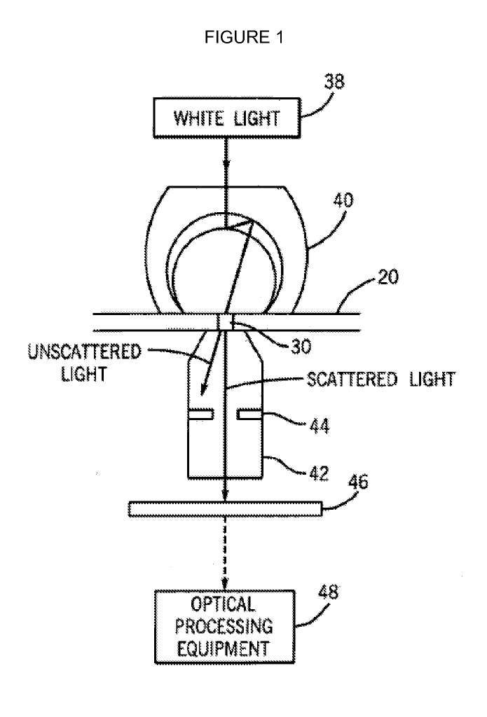

A dark field microscopy instrumentation setup for detecting, observing, and measuring the scattered light from gold nanorod 30 is shown in FIG. 1. A full spectrum white light from light source 38 is incident to dark field condenser and lens 40, which in turn alters the path of the light to create an oblique angle with respect to the long and short axes orientation of gold nanorod 30. The F1-ATPase enzyme 10 with attached gold nanorod 30 is positioned on slide surface 20. The F1-ATPase enzyme 10 and rotating gold nanorod 30 are exposed to the white light. The rotational motion of the F1-ATPase enzyme 10, and the corresponding rotation of gold nanorod 30, cause the red and green wavelengths of the incident light to scatter depending on the orientation of the nanorod. Red light scatters when the long axis is exposed to the white light and green light scatters when the short axis is exposed to the white light. The unscattered light continues on into objective lens 42 where it is blocked by iris 44. The wavelengths of the light, which are scattered by gold nanorod 30 pass through iris or aperture 44.

A polarizing filter 46 is positioned at the output of objective lens 42 and passes the wavelength of the scattered light which is aligned with the polarizing filter, and further blocks any light, scattered or not, which is not aligned with the polarizing filter. The scattered red light, which is aligned with polarizing filter 46, passes through the filter. Likewise, the scattered green light, which is aligned with polarizing filter 46, passes through the filter. The intensity of the red and green scattered light varies relative to the angle of polarization and has a maximum when the appropriate axis of gold nanorod 30 is parallel to the plane of polarization. When the light scattered from the long axis of gold nanorod 30 is aligned with polarizing filter 46 then red light is passed. When the light scattered from the short axis of gold nanorod 30 is aligned with polarizing filter 46 then green light is passed. No other light passes through polarizing filter 46, i.e., the output of polarizing filter 46 is otherwise dark.

The red and green light intensities are collected by optical processing equipment 48, which separates the red and green light into individual channels. MetaVue software is used to isolate, detect, observe, and measure the intensity of the red and green light. The F1-ATPase enzyme-induced rotation is observed as red and green blinking light through polarizing filter 46, due to surface plasmon resonance of gold nanorod 30. The alternating blinking red and green light has provided an observable and quantifiable representation of the physical and behavioral characteristics of the F1-ATPase enzyme 10, e.g., the blinking rate of the red and green light is a function of the speed of rotation of the F1-ATPase enzyme 10.

The nanoparticles can be ellipsoidal, rod-shaped, or other anisotropic shapes. The nanoparticles can be made of pure metals or alloys, and can be coated with a different type of metal or other material like glass. The nanoparticles can be flat structures patterned onto a metalized surface, for example by means of e-beam lithography.

Inducing movement of the molecular motor is done by standard methods in the art for a given molecular motor. For example, the movement of F1 ATPases is induced by adding ATP using standard techniques (Noji, H., Yasuda, R., Yoshida, M. and Kinosita, K. (1997) Nature 386, 299-302). Suitable concentrations of ATP for use in the methods of the invention range from 1 pM to 2 mM; preferably between 200 μM and 1 mM The rate of rotation of the FI-ATPase can be controlled by the ATP concentration used. For example, some detection methods are capable of detecting greater rates of rotation than others, and thus the specific concentration of ATP used will depend in part on the detection technique to be employed.

Those of skill in the art are able to determine how to induce movement of other known molecular motors using similar published protocols. It is a specific aspect of the present invention that the only motion that will be detected will result from molecular motors that are connected to the detection probe and this connection is dependent on, and occurs only if, the small molecule metabolite to be detected being bound to the protein in the bridging part of the molecule. Since that connection will depend upon the presence of the small molecule metabolite to be detected being bound to the protein in the bridge resulting from specific binding of the small molecule metabolite to be detected and the protein, observation of this motion will identify the presence of the small molecule metabolite to be detected.

Detecting movement of the molecular motor through the detection probe can be accomplished by any suitable means. In one embodiment, direct visualization of the movement is used. In a preferred embodiment, elemental metal rod detection probes capable of visual observation by microscope are attached to the bridging portion of the device.

Other means of observation include, but are not limited to single molecule fluorescence resonance energy transfer, fluorescence lifetime anisotropy, and atomic force microscopy.

Beside microscopy, other methods can be used to observe the rotation of detection probe, including but not limited to (1) attaching the molecular motor onto a nano-electrode and measure the micro current change or impendence change produced by rotation; (2) attaching a fluorescent label such as Pacific Blue™ (Molecular Probes) on a non-rotating part of the molecular motor; and (3) single molecule anisotropy measurement. In another alternative, rotation can be observed through periodic quenching of the fluorescence signal by a quencher detection probe. In a further alternative, a surface plasmon resonance biosensor can be used to measure the surface plasmon resonance change during metallic nanorod rotation.

In a most preferred embodiment, metal (such as gold) nanorods are used with visible light (400-700 nm wavelength range) to detect the rotation, to provide improved detection capability (See, for example, WO 2004/053501). The light scattered from the nanorods is polarized with the longer and shorter wavelengths scattered from the long and short axes, respectively, of the rod. When viewed through a polarizing filter, the intensity of scattered light depends on the angle of the rod relative to the direction of the filter. The light scattered from the long and short axes of the rod is observed to have a maximum value when those axes are parallel to the direction of the filter and a minimum when perpendicular to the filter.

For example, if the long and short wavelengths of scattered light are red and green, respectively, the intensity of the red will be maximum when the green is minimum. Thus, rotation of a metal nanorod viewed through a polarizing filter will appear to blink red and green. In this embodiment, monitoring the oscillation of intensity of both the red and green light as the nanorod rotates provides independent conformation that the rod is rotating. In a further preferred embodiment, the oscillation of intensity of light of only one wavelength is measured, which further improves signal to noise ratios. In these embodiments, measurements can be made using both wavelengths (using, for example, a beamsplitter or a color camera) or just one wavelength of light (using, for example, a green or red filter).

Some digital cameras are limited with regard to the frame rate (speed of data collection) at which the camera is still sensitive enough to measure the intensity oscillations from the rotating nanorods. Single photon counters can be used to make the oscillation measurement.

The pin hole acts as a camera obscura and the oscillation of only one rod at a time can be measured; it is capable of much greater frame rates at much higher signal to noise. Digital cameras can collect oscillation data on many nanorods at once, while the speed and sensitivity of the camera only needs to be sufficient to capture the rate of rotation of the rod.

The preferred oscillation rate is one that is easily measured with the detection device used to make the measurement. In these embodiments employing elemental metal nanorods, dark field microscopy is the preferred detection method, because only the light scattered off the nanorods is observed, further improving signal to noise ratios. In another embodiment, detection is performed using light field microscopy.

Since the methods of the invention are capable of detecting single molecules of the small molecule metabolite to be detected, they provide a precise means to quantify the amount of the small molecule metabolite to be detected present in a sample. In one embodiment, the number of rotating molecules is determined by visualization and a calculation is made for the fraction of the total sample that is being viewed. This is not possible with fluorescent detection methods in current use with DNA microarrays.

While in specific embodiments the anchoring part is linked to the adjacent bridging part through a biotin/avidin, other binding pairs may be used to achieve this linkage. Non-limiting examples of other such binding pair examples include digoxigenin (DIG)/anti-digoxigenin antibody and other antigen/antibody pairs. Epitope tags, such as a his-tag, and antibodies directed against the epitope tag (or fragments thereof) are further examples of binding pairs for use with the methods of the present invention. Those of skill in the art will understand that certain embodiments listed herein as indirect binding of the affinity tag and the molecular motor or detection probe can also be used for direct binding embodiments.

### C. THE BRIDGING PART

In the present invention, in between the nanoparticles and the gamma subunit there is a linking, bridging or coupling portion that is comprised of a protein that binds to or otherwise responds to the presence of a metabolite to be detected. As noted herein above, the protein may be a transcriptional regulator protein, a signal transduction protein, a DNA proofing protein or indeed any other protein that acts as binding protein for a metabolite of interest.

Exemplary transcriptional regulator proteins include JNK1 (C-jun kinase 1; mitogen-activated protein kinase 8; MAPK8); P38 kinase (mitogen-activated protein kinase 14; MAPK14; p38 MAP KINASE; p38-ALPHA); ATF2, MEF2C, and MAX, cell cycle regulator CDC25B, the tumor suppressor p53; gene encoding cdc2, a gene encoding Cyclin (Cyclin A, Cyclin D, Cyclin E), cdc25C, WAF1 (wildtype p53-activated fragment 1), INK4, CDK (CDK1, CDK2, CDK4, CDK6), Rb protein, and E2F. Exemplary transcription repressor proteins include, e.g., tetracycline (tet) repressor protein. As discussed above, a specific signal transduction protein that could be used is protein kinase A. Other protein kinases that may be used include Ras, A-Raf, B-Raf, and Raf-1 and the like.

### D. KITS AND EXEMPLARY EMBODIMENTS

In another aspect, the present invention provides kits for detection of a small molecule metabolite comprising (a) a protein that specifically binds the small molecule metabolite of interest, wherein the protein is biotinylated in a manner that does not interfere with the binding of the small molecule metabolite of interest; (b) a molecular motor such as a 3 subunit or 5 subunit F1-ATPase molecule wherein the F1-ATPase molecule has a his-tag to facilitate attachment of the F1-ATPASE to a solid support and further wherein the F1-ATPase is biotinylated; (c) gold nanorods bound to molecules that recognize the protein in (a) when the protein is bound to a small molecule metabolite of interest. The kits may further comprise a solid support, and avidin.

In preferred embodiments, the kit further contains a molecular motor that binds to the bridging part and/or a detection probe that binds to the bridging part. In a further embodiment, the molecular motor is bound to a solid support, such as a glass coverslip or other suitable support. The support can be derivatized in any manner suitable for binding to the molecular motor.

The present invention also provides a composition comprising an anchoring part, a bridging part and a signal producing part, wherein:
(a) the anchoring part comprises a F1-ATPase molecule modified via site directed mutagenesis so as to comprise a his-tag on the N-terminus of an F1-α or F1-β subunit and a cysteine on the F1-γ subunit wherein a plurality of said F1-ATPase molecule are affixed to the surface of a microscope slide such that each F1-ATPase molecule is oriented with the F1-γ subunit away from the surface of said microscope slide, wherein said cysteine on the F1-γ subunit is biotinylated;
(b) the bridging part comprises a protein that binds to or otherwise responds to the presence of a small molecule metabolite to be detected, wherein said protein in said bridging part is biotinylated and linked to said anchoring part through a biotin-avidin-biotin linkage with said biotinylated F1-γ subunit of said anchoring part; and
(c) the signal producing part comprises an electromagnetic detection probe that has been functionalized with moieties that specifically bind to said protein of said bridging part in the presence of said metabolite to be detected.

The present invention also provides a composition comprising: an anchoring part, a bridging part and a signal producing part, wherein:
(a) the anchoring part comprises a F1-ATPase molecule modified via site directed mutagenesis so as to comprise a his-tag on the N-terminus of an F1-α or F1-β subunit and a cysteine on the F1-γ subunit wherein a plurality of said F1-ATPase molecule are affixed to the surface of a microscope slide such that each F1-ATPase molecule is oriented with the F1-γ subunit away from the surface of said microscope slide, wherein said cysteine on the F1-γ subunit is biotinylated;
(b) the bridging part comprises a transcriptional regulator protein that comprises a binding site for a specific DNA sequence when the protein is bound to or otherwise responds to the presence of a small molecule metabolite to be detected, wherein said protein in said bridging part is biotinylated and linked to said anchoring part through a biotin-avidin-biotin linkage with said biotinylated F1-γ subunit of said anchoring part; and
(c) the signal producing part comprises an electromagnetic detection probe that has been functionalized with specific DNA sequences known to bind said transcriptional regulator of said bridging part when the protein is bound to said metabolite to be detected.

The present invention further provides a composition comprising: (a) a solid support; and (b) a plurality of molecular motors attached to the solid support, wherein the plurality of molecular motors are attached to a bridging part that comprises a protein that is specific for the small molecule metabolite to be detected. In a preferred embodiment, the plurality of molecular motors comprises more than one type of molecular motor. In a further preferred embodiment, the different types of molecular motors on the support comprise different proteins in the bridging part that are specific for different small molecule metabolites to be detected. In a further preferred embodiment, the composition further comprises a first protein in the bridging part that is a transcriptional activator protein that specifically binds the small molecule metabolite to be detected bound to a molecular motor through a biotin-avidin-biotin linkage. In a further preferred embodiment, the protein in the bridging part is a transcriptional repressor protein that is bound to a DNA molecule that is bound to gold nanorods through a linkage with an affinity tag such as a biotin-avidin linkage.

Such methods are of value, since gold nanorods are preferred for use in detection assays, such as those described herein, for the reasons discussed above.

### F. EXAMPLES

### Example 1

In one embodiment of the present invention, a first affinity tag biotin is attached to a cysteine residue on a protein that will form a part of the bridging part of the device of the present invention. This biotinylated protein is used as a bridge between a molecular motor and the detection probe used to detect the motion generated from the motor via the affinity tags. The biotinylated protein of the bridging part is attached to the moving component of the molecular motor. The bridging part becomes connected to the detection part when the target small molecule metabolites to be detected are present because the conformation of the protein in the bridging part is altered such that the moiety that is present on the detecting probe is able to bind to the bridging part of the nanodetection device. In the specific example, the detection probe comprises gold nanorods that can be visualized by microscopy, attached to the moiety that binds to or recognizes the protein in the bridging part is bound to the nanorod by a biotin avidin bond. The particular gold rods were of a size and were illuminated such that regularly changing color change from red to green (or black, if red only) and back, characteristically indicated rotation of the rods.

After assembly of the F1-ATPase, the bridging part and preparation of the nanorod detection part, the F1-ATPase is immobilized onto a solid substrate such as a microscope slide. This immobilization is effected by histidine binding of the nonrotational F1 motor structure to a nickel surface.

The movement of the molecular motor is induced by adding ATP. The only motion that will be detected will result from motors that are connected to the attached detection probe. Since that connection will depend upon the presence of the target small molecule metabolites to be detected being bound to the protein in the bridge and because binding of the moiety on the nanorod bead to the protein in the bridging part can only result when the target small molecule metabolites to be detected is bound to the protein in the bridging part, the observation of this motion will identify the presence in the sample of the target small molecule metabolites to be detected.

### Example 2

Four components of a nanodetection device were prepared separately: (1) a protein bridge made from the specific protein for the target small molecule metabolites to be detected; (2) modified F1-ATPase; (3) nickel-coated coverslips; and (4) coated nanorods. After these components were prepared, they were assembled into the device by adding the target small molecule metabolites to be detected. Rotation of the nanorod attached to the device was observed only if the target small molecule metabolites to be detected was present to enable assembly of the device.

The protein that will serve as the bridge protein will be genetically modified to incorporate a cysteine in a location that will be in a location that is optimal for attachment to the molecular motor via a biotin-avidin-biotin linkage. The location of the cysteine will be chosen specific to the bridge protein to insure that the functional characteristics of the bridge protein are not altered, and that the cysteine does not interfere with the formation of the DNA recognition/binding site that will be used to attach the visible probe. The bridge protein may also be genetically modified to change its specificity for the target molecule. In one example, mutations may be made to the arabinose activator protein to increase its affinity for glucose in lieu of arabinose. In another embodiment, the DNA recognition/binding site may changed by genetic modification of the protein so that the bridging component recognizes a different DNA sequence to complete assembly of the nanodevice.

Modified F1-ATPase.

The FI-ATPase isolated from E. coli strain AB004 contains all five subunits with the stoichiometry α3β3γδ∈. The sequences of these proteins correspond to gene bank accession numbers: α, AAA24735; β, AAA24737; γ, AAA24736; δ, AAA24734; and ∈, AAA24738 with the following changes. Mutations were made to replace all existing cystines in the α, β, γ, δ and ∈ subunits with alanines. The α subunit was mutated to extend the N-terminus with 6 histidines. The γ subunit was mutated to replace lysine-109 with a cysteine that served as the site of biotinylation using biotin maleimide. Biotinylation was carried with a 3-fold molar excess of EZ LINK™ PEO maleimide activated biotin (Pierce Endogen; product #21901) by incubation with gentle shaking for one hour at room temperature. Unbound biotin was removed by size exclusion gel filtration. The biotin covalently bound at this site serves as an effective binding site for avidin, to which other biotinylated moieties can be attached including the bridge components like the metabolite activating proteins.

The placement of this cysteine can be varied on the αβ domain of the γ subunit or on ∈ subunit to any exposed location that does not interfere with ATPase activity and/or rotation. The three subunit α3β3γ subcomplex of the enzyme can also serve as the F1-ATPase, as can the five subunit F1-ATPase in which the 6 subunit cysteines were replaced with alanines by mutagenesis. The 3 subunit or 5 subunit FI-ATPase purified from any biological source will suffice for this task as long as the his-tag and cysteine modifications are made. The his tag used to attach F1 to the coverslip can alternatively (or additionally) be on the α and/or β subunits as long as it does not interfere with ATPase activity. An α3β3γ subcomplex of the FI ATPase from the thermophilic bacterium PS3 that contained a 10×his tag on the β subunit and a cysteine in a location that facilitated biotinylation and DNA attachment was also used successfully in similar experiments. Similar working construct can also be made using F1, for example, from Chlamydomonas or spinach chloroplasts.

Biotinylated F1 was added to 500 μl of washed nickel-nitrilotriacetic acid (Ni-NTA) affinity resin (Sigma-Aldrich product # P6611), and stirred gently for 30 minutes at room temperature to allow binding of the 6×His-tagged F1 to the Ni-NTA resin. The Ni-NTA resin with bound F1 was loaded into a syringe-column and the column was flushed with 1 ml of a washing buffer. Neutravidin (Molecular Probes product # A2666) was dissolved in wash buffer at a concentration of 1 mg/ml and an approximately 8 to 10-fold molar ratio, relative to the initial F1 concentration, was passed over the Ni-NTA resin to allow binding of Neutravidin to the biotin moieties. Following Neutravidin treatment, the Ni-NTA resin was flushed with 5 ml of washing buffer to remove unbound Neutravidin, then the biotinylated and avidinated F1 was released and collected from the column with 1 ml of an elution buffer.

Binding the avidin in this manner to the F1 allows a large excess of avidin to be used and ensures that all the F1 has bound avidin. Any F1 that remains without avidin will decrease the sensitivity of detection. If the avidin is added to the F1 after the FI has bound to the cover slip, this allows the biotinylated protein of the bridging part to bind directly to the coverslip in the absence of F1. Such bound protein will bind a nanorod but will not be able to rotate, and thus decreases the sensitivity and be counted as increased background.

Following the biotinylation of F1 and gel filtration steps, the recovery of F1 is typically 5% of the starting amount. Following binding of biotinylated F1 to Ni-NTA resin, avidination, and elution from the Ni-NTA resin, approximately 75% of the starting F1 was recovered. ATPase activity of biotinylated and avidinated Fi was approximately 90% of the initial activity.

Procedure for Preparing Ni-NTA Cover Slip.

Ni-NTA cover slips were made following a procedure by Kastner et al. (2003, Biophysical Journal 84: 1651-1659). The cover glasses (22×22 mm, VWR,) were precleaned by baking at 500° C. for 2 hours. Successively, the glasses were incubated in sealing solution (2% (v/v) 3-glycidyloxypropyl-trimethoxysilane (Fluka, Buchs, Switzerland), 0.01% (v/v) acetic acid) for 3 h at 90° C.; coating solution (2% (w/v) N,Nbis(carboxymethyl)-L-lysine (Fluka, Buchs, Switzerland), 2 mM KHCO3, pH 10.0) for 16 h at 60° C.; and Ni2+ solution (10 mM NiSO4, 5 mM glycine, pH 8.0) for at least 2 h at room temperature. After each coating step the glasses were washed with ultrapure water.

Protocol to Synthesize Gold Nanorods and Coat them with Avidin.

The factors affecting shape and size of gold nanoparticle include the concentration of cetyltrimethylammonium bromide (CTAB), Au seed concentration, presences of silver (AgNO3), NaOH, ascorbic acid concentration, and appropriate combinations of all these factors. Techniques to increase the percentage of gold nanorods relative to other shapes are of value since gold nanorods are preferred for use in detection assays, such as those described herein. In a typical experiment to synthesize gold nanorods, the CTAB-coated seed solution was prepared by adding 25 μl of Au solution (50 mM) to 10 ml volume of CTAB (100 mM), then 55 μl of NaBH4 (30 mM, ice-cold) was added with strong vortexing for about 2 min. This seed solution can be used immediately, but will keep active at least for one day.

To grow gold nanorods, 100 μl of Au solution (50 mM) was added to 10 ml CTAB (100 mM), followed by adding AgNO3 (10 mM, 50-125 μl) with gentle shaking. Then, 85 μl of ascorbic acid (100 mM) was added with immediate shaking, rendering the solution colorless. Finally, 24 μl of prepared seed solution was added with gentle shaking. A mixture of violet and blue colors appear within 10-20 minutes. Growing gold nanorods at high temperatures (55˜100° C.) resulted in more of the deep blue color in the solution. In this method, the seed concentration is the most important parameter for increasing the percentage of nanorods.

Gold avidination procedure: 1.5 ml of gold rod preparation is used; this is centrifuged for 10 min at 4000 rpm; the supernatant is removed and the pellet is resuspended in 1 ml of 1 mM CTAB. This mixture is centrifuged for 5 min at 6000 rpm, the supernatant removed and the pellet is again resuspended in 0.5 ml of 1 mM CTAB. An absorbance spectrum is taken and the sample is diluted with 1 mM CTAB so that the rod absorbance peak (A650) is approximately 2.0. Neutravidin is added to a final concentration of 40 ug/ml; and the mixture is incubated at room temp with light agitation for 1 hr. This produces avidinated rods can be stored at room temp or 4 degrees C.

Assembly of the Components into a Functional Device.

5 μl of avidinated F1 (80 g/ml) in Tris buffer (50 mM Tris, 10 mM KC1, pH 8.0) is spotted onto a Ni-NTA coverslip and incubated for 5 minutes. The coverslip is then washed for 30 sec with Tris buffer. The next step involves adding 4 μl biotinylated protein bridges to the coverslip and incubating for 5 min. The assembly is then washed for 30 sec with Tris buffer. The assembly is then incubated with 4 μl avidin coated gold rods in the presence of a small molecule that will bind to the protein of the bridging part of the assembly. The mixture is incubated for 5 minutes and washed for 30 sec with Tris buffer. To view rotation under the microscope, add 4 μl of Tris buffer. This buffer will also contain 1 mM Mg2+-ATP to induce rotation.

The volume of each of the components added to the coverslip can be varied as desired based on the assay format. Although this example shows addition of 1 mM Mg²⁺-ATP to induce rotation, as little as 0.4 mM Mg²⁺-ATP has been added and has still shown rotation. At these lower ATP concentrations, the rate of rotation is slower and thus the frame rate needed to record the rotation does not have to be as fast. Rotation may be observed at ATP concentrations as low as 2 pM using the 3 subunit subcomplex of F1 from the thermophilic bacteria PS3. An alternate protocol to assemble the device using the protein bridge with biotin and DIG such that the DIG would bind to anti-DIG coated nanorods is described below. This experiment also uses an FI that differed from the one described above in that it contained an additional mutation of γY215C for biotinylation, so that avidin can make a two point attachment to F1. This experiment differs from that described above in that it employs a flow cell. Consequently, a movie can be made of the assembled devices before the addition of ATP, then again after ATP is added to induce rotation.

45 μl of 0.5 nM F1 with a subunit composition of α3β3γδ∈ and including mutant (γK109C, γL215C) was mixed with 8 μl of protein for forming the bridge. The mixture is incubated at room temperature for 30 minutes. An equal volume of BSA (20 mg/ml) was then added to the sample. Microscope flow cells are prepared by attaching an Ni-NTA cover slip to a glass slide using double-sided Scotch tape. Each flow cell is filled with 25 μl of sample and incubated at RT for 5 min. The flow cells are washed with 300 μl washing buffer (50 mM Tris, 10 mM KCl, pH 8) containing 10 mg/ml BSA. 25 μl of anti-DIG coated gold nanorods were added and incubated at RT for 5 min, followed by 5×200 μl washes with washing buffer. 100 μl of rotation buffer (50 mM Tris, 10 mM KC1, pH 8, 0.2 mM ATP, 0.2 mM MgCl2, 29.1 mg/ml phosphoenolpyrivate (PEP), 1.25 mg/ml pyruvate kinase (PK), 1.25 mg/ml lactic dehydrogenase (LDH), 4 mg/ml reduced nicotinamide adenine dinucleotide (NADH)) was added to the flow cell before putting the flow cell under the microscope. The PEP, PK, LDH and NADH were used to regenerate ATP from ADP and phosphate so as to keep the ATP concentration constant throughout the measurement. Movies can be taken at 500 frames per second with or without beam splitter. The beam splitter allows the measurement of the oscillation of the intensity of both the red and green scattered light from the nanorod in each frame of the movie. In the absence of the beam splitter a red filter was used to measure the oscillation of the red only. The analyses of the oscillations are described below.

Analysis of the Oscillation of Intensity of Light Scattered from a Nanorod to Determine if it is Rotating.

One-Step Detection Procedure.

Slides are prepared with a fully assembled the nanodetection device and with ATP present in solution. In order to be able to distinguish Brownian motion from actual rotation, it is preferred to gather data at a rate fast enough to be able to separate the two sources of variation. In a preferred embodiment, this is accomplished using a single photon counter to measure the variation, which permits rotation visualization in real time.

Multistep Procedure Another way that rotation can be detected is to use a flow cell and a high speed video camera. This requires taking at least two movies, although three is preferred. At a minimum, movies are made of a rod in both the presence and absence of ATP. It is helpful to have a movie of the rod while the polarizing lens rotates. This determines the depth of the oscillation of intensity to be expected if that rod is rotating. This control increases the confidence in detecting F1 dependent rotation, however it can be confirmed without the extra measurement.

Software: Two different software platforms that have been developed to achieve the same goal, is the rod rotating due to the F1 or not. The data collected is always the intensity as a function of time.

The first method involves analyzing trends in the data from the high speed camera.

The software reads the data, preferably from three experiments: without ATP, without ATP while rotating the polarizer, and with ATP. The dynamic range expected during rotation is calculated from the polarizer control and can be used to ensure that the molecule being examined is actually a gold rod. The dynamic range is then compared to the standard deviation from the measurements with and without ATP. If the movie with ATP is different than the one without ATP, and fluctuates through the full dynamic range calculated, then the molecule is rotating. The software program is able to do this for all rods in the given field of view. The software is the preferred way of determining rotation, however it is possible to collect and interpret the information by hand to make a final determination. Either way the criteria are the same. The software can be implemented using standard techniques by those of skill in the art. The software is specific to answering the question of whether rotation is occurring or not for all of the rods found in the field of view.

The software developed for the photon counting system calculates numerous statistical measures common in signal processing, including the period, frequency, transition rate, duty cycle, over shoot, under shoot, dwell time, Fourier transformation, and power spectrum. The only required statistic is the power spectrum, although the Fourier transformation is generally calculated to get the power spectrum. From the power spectrum one can determine if there is rotation occurring that is distinct from Brownian motion. It is useful to have the other statistics from additional analysis of the motion, but they are not required to determine if F1 dependent rotation occurs. The software can be implemented using standard techniques by those of skill in the art.

### Example 3

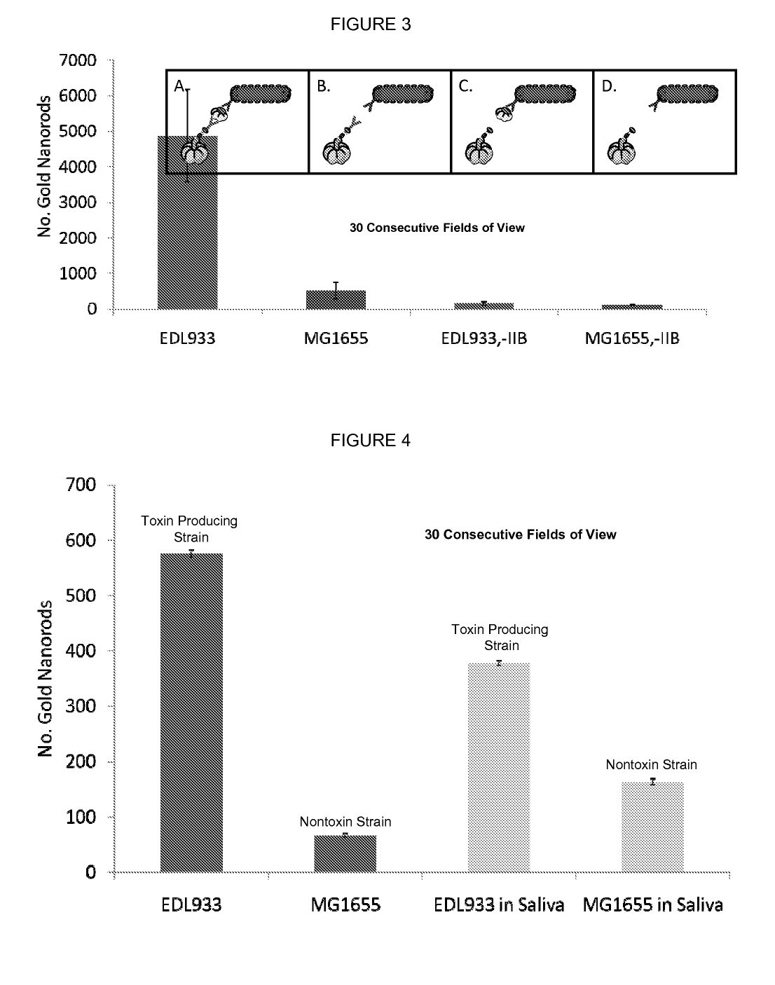

In some embodiments of the present invention, the nanodetection device is used to detect protein targets. In one example, the Stx2 Shiga toxin produced from E. coli O157:H7 strain is used as a protein target. Shiga toxin-producing E. coli (STEC) strains produce cytotoxins known as Stx1 and Stx2 Shiga toxins, which have a subunit composition of A,B₅. To detect Stx2 toxin, anti-Stx2A and anti Stx2B monoclonal antibodies are used as the detection and capture antibodies, respectively in a nanodevice. For protein detection, biotinylated-Protein G is bound to avidin attached to the F₁ motor on the slide. The Protein G binds and immobilizes the anti-Stx2B (“capture”) antibodies on the surface of the slide. Gold nanorods are coated with non-biotinylated Protein G to which the “detection” antibody, i.e. anti-Stx2A, is bound. The detection and capture antibody are specific to different domains of the target protein (Stx2 toxin). This nanodevice can detect Stx2 toxin in cellular supernatants containing the Stx2 producing strain of E. coli (EDL933) (FIGS. 3A and 3C) as compared to an E. coli non-toxin producing strain (MG1655) (FIGS. 3B and 3D) by determining the number of nanorods observed in 30 consecutive fields of view. FIGS. 3C and 3D are controls; in FIG. 3C the capture antibody was omitted and in FIG. 3D the Stx2 toxin was omitted.

The nanodevices can detect Stx2 toxin protein in saliva samples compared with purified toxin containing the toxin producing strain (EDL933) or non-toxin strain (MG1655) as measured by the number of nanorods observed in 30 consecutive fields of view as shown in FIG. 4.

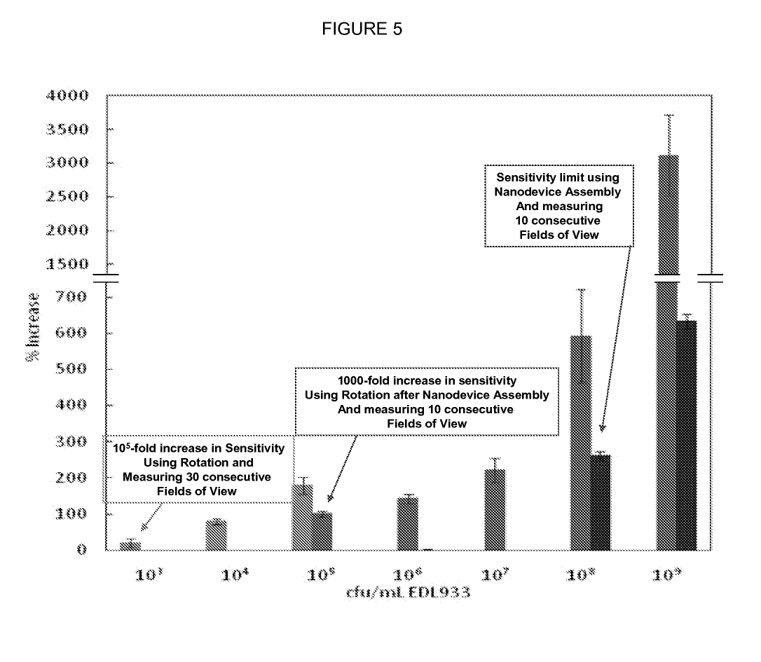

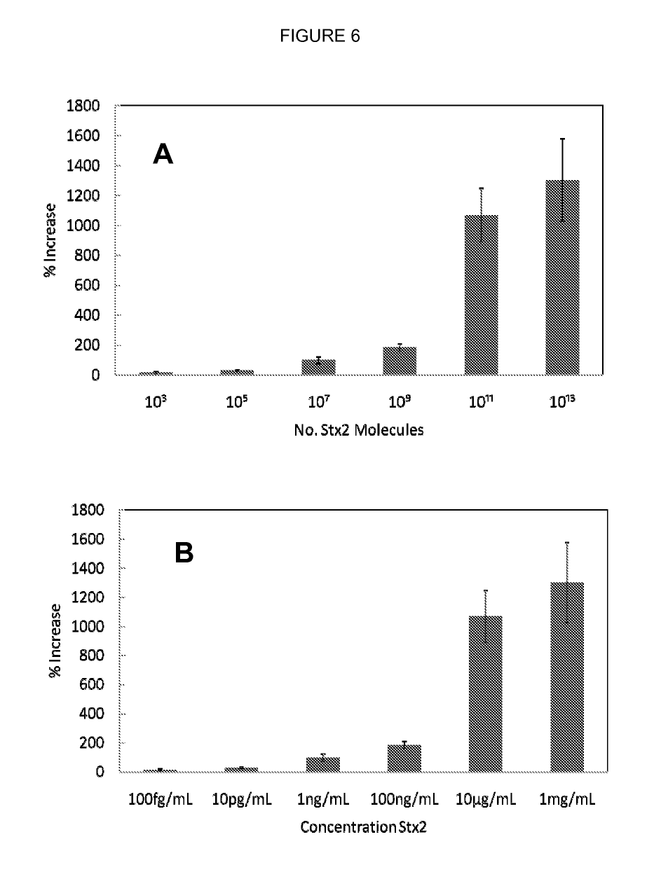

FIG. 5 shows the detection limit for detecting Stx2 protein toxin in crude cell supernatants presented as cell forming units (i.e. the number of bacterial cells) of the toxin-producing strain per ml. Detection was measured in 10 consecutive fields of view by nanodevice assembly (blue bars) versus the number of rotating nanorods in 10 (red bars) or 30 (green bards) consecutive fields-of-view. A detection limit of 10⁸ cfu/ml was obtained by counting the number of assembled nanodevices relative to nonspecifically bound nanorods in 10 consecutive fields of view. This detection limit was improved by 1000-fold by counting the number of rotating nanorods in the same number of fields-of-view, and was improved by 100,000-fold to a limit of 10³ cfu/ml by measuring rotation in 30 consecutive fields-of-view. The rotation-based detection limit of the system using purified Stx2 target protein is shown in FIG. 6. The data is expressed as a function of the number of target molecules present (FIG. 6A) or the target concentration (FIG. 6B) in the samples. Using 2 μl sample volume and counting only 10 fields-of-view, as few as 1000 target molecules can be detected in 10 fg ml⁻¹ solution.

### Example 4

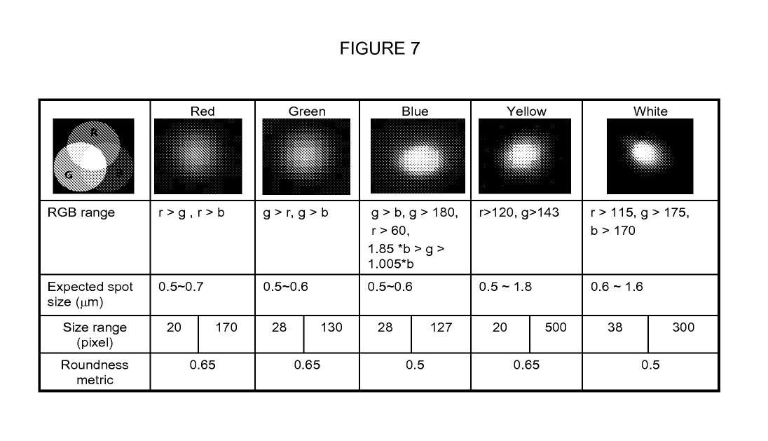

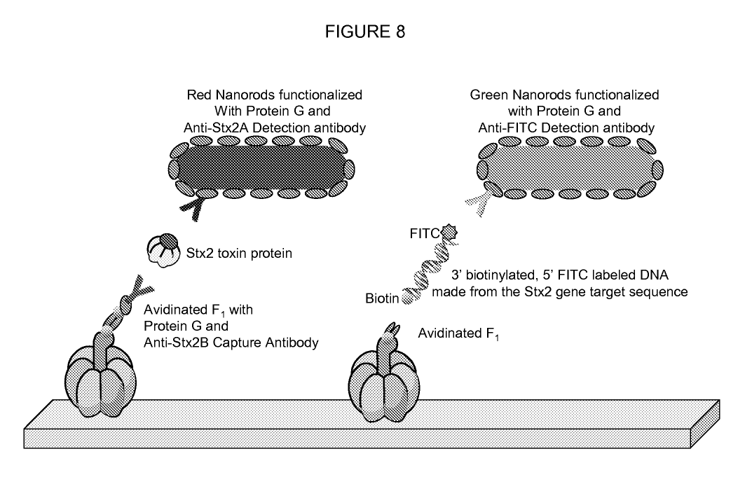

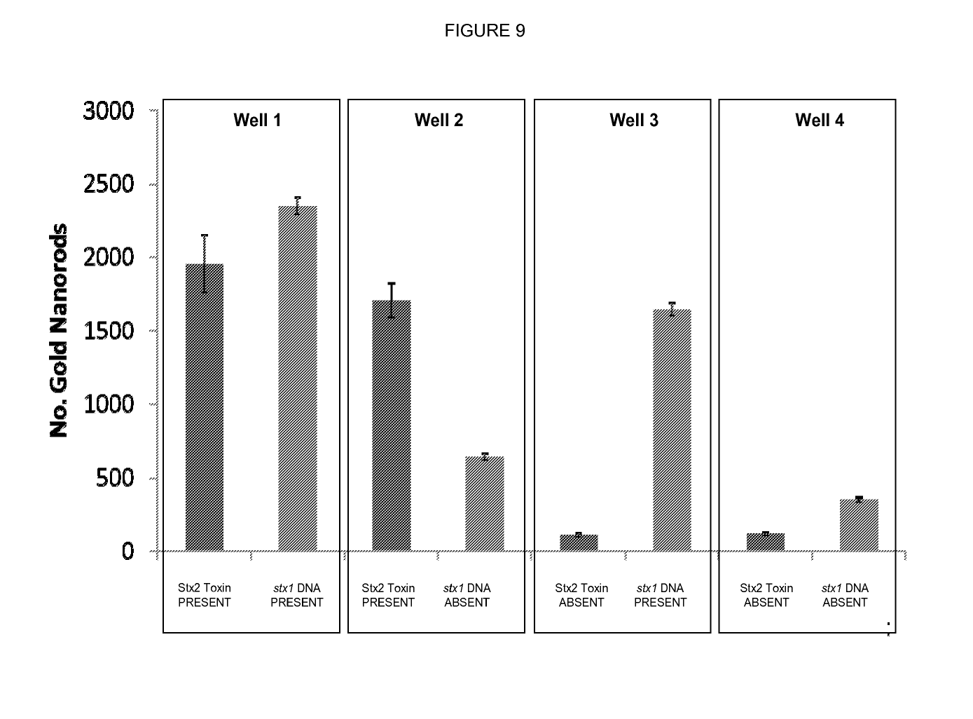

In some embodiments of the present invention, nanodetection devices are used to detect more than one target in a sample. In some embodiments, multiple targets can be detected in a single sample, for example a protein target and a DNA target. This can occur by using different color nanorods, e.g. green, red or yellow, to correspond to the different target molecule. FIG. 7 depicts the summary of criteria used in the algorithms employed to identify and quantify the number of red, green, blue and yellow nanorods in a digital photo of a field of view. These include, the number of adjacent pixels within a given range of red, green and blue values as well metrics that quantify the roundness and diameter of the adjacent pixels within a common color range that are in a given range with expected values for a nanorod of a given color. Only groups of consecutive pixels that are sufficiently round and have a common range of red, green, blue (RGB) values that correspond to their diameter are counted as a nanorod of a specific color, thus light from nanoscale dust is removed. FIG. 8 depicts the components for nanodevices for multiplex detection in a single well for the Stx2 toxin protein (red nanorods) and the stx2 gene DNA sequence (green nanorods). The slide surface in each well contained F₁ that was modified in one of two ways. One filed of F₁ molecules contained an exposed avidin for DNA detection and the second superimposed filed of F₁ contained an exposed capture antibody for the Stx2 protein (anti-stx2B antibody). The samples of supernatant from E. coli O157:H7 were incubated in the wells followed by the addition of a solution containing nanorods. Red nanaorods were functionalized with Protein G and anti-stx2A antibodies and green nanorods were functionalized with protein G and anti-FITC monoclonal detection antibodies. When stx2 gene target was present, it was converted to 3′ biotinylated, 5′ FITC-labeled DNA for nanodevice assembly. FIG. 9 depicts the results of the simultaneous multiplex detection of protein and DNA for the Stx2 toxin and stx1 gene as an average of 3 replications. The data shows that specific protein and DNA targets can be detected and distinguished from each other when probed simultaneously in the same well.

### Example 5

In some embodiments of the present invention, a nanodetection device is used for metabolite detection comprising a transcriptional activator protein as an actuator. One embodiment is a device that can detect a single molecule of cyclic adenosine monophosphate (cAMP). cAMP is known as a second messenger because its synthesis is regulated by a range of signaling molecules through the activation and inhibition of adenylyl cyclase stimulatory and inhibitory G-protein-coupled receptors. The bacterial protein, Catabolite Activator Protein (CAP), which binds to cAMP to form an active conformation will be used in the nanodevice. CAP bound to cAMP bind to DNA that is involved in the positive regulation of lac operon.

This bacterial Catabolite Activator Protein (CAP), preferably E. Coli CAP, will be modified to contain a cleavable his tag (for purification purposes) and a cysteine (for biotinylation by biotin maleimide) in a location that will allow assembly with the F1-ATPase rotor via the binding of avidin. The cysteine may be added, for example, but not limited to, the N-terminus or residue-109 of E. coli CAP. The DNA molecules containing the sequences that enable binding to the activated conformation of CAP will be biotinylated for attachment to avidin-coated gold nanorods.

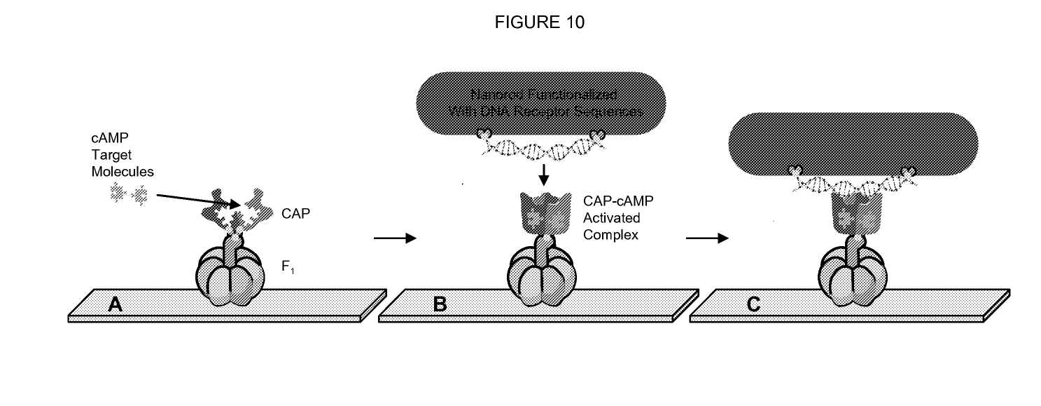

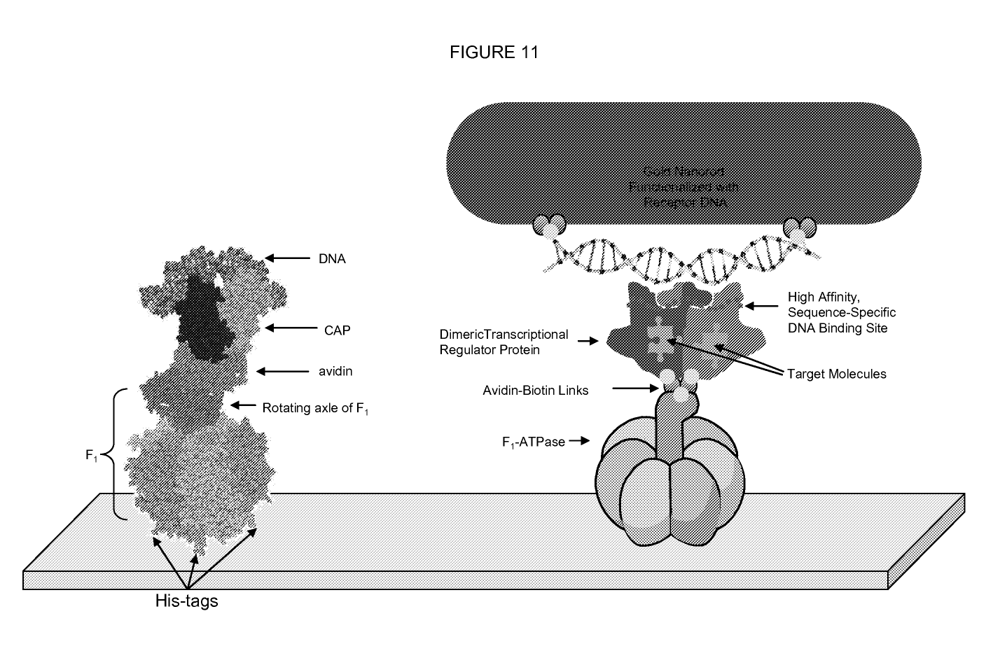

The nanodevice comprises a F1 molecular motor, the altered CAP and a gold nanorod functionalized with DNA receptor sequences, as depicted in FIG. 10. Detection of target cAMP that activated assembly of the nanodevice will be accomplished via dark field microscopy of light scattered from the nanorods.

Binding of the cAMP to CAP can induce a conformational change that creates a high affinity binding site for the DNA receptor sequence. The microscope slides will be prepared to contain an array of bound biotinylated F₁ to which biotinylated CAP will be immobilized via biotin-avidin linkages. The high affinity binding of target cAMP molecules to the CAP immobilized on the rotating arm of the F₁ molecular motor on the slide (FIG. 10A) will activate the protein to create a high affinity binding site for its receptor DNA sequence (FIG. 10B). Assembly of the nanodevices is then completed by addition of gold nanorods functionalized with a coating of the DNA molecules with the sequence that specifically binds to the activated CAP-cAMP complex (FIG. 10C). Detection of target cAMP that activated assembly of the nanodevices will be accomplished via dark field microscopy of light scattered from the nanorods as described herein.

### Example 6

In some embodiments of the present invention, metabolite detection is accomplished using a transcriptional repressor protein. In one embodiment, a nanoscale detection device to detect single molecules of NADH is made using the NADH repressor protein (NRP).

Reduced nicotinamide adenine dinucleotide (NADH) is a major form of cellular energy currency. Mitochondria oxidize NADH to make ATP during oxidative phosphorylation, the levels of cellular NADH can provide insight into the cellular energy reserves. NRP is a well characterized transcriptional repressor protein.

NRP will be expressed from a plasmid that contains NRP with a cleavable his-tag (to provide a form that can be purified using nickel beads). The NRP sequence will also be altered to include a cysteine that does not interfere with NRP function but can be biotinylated via biotin maleimide. NPR will be immobilized on the rotating arm of the F₁ molecular motor via avidin. Nanorods functionalized with avidin will be coated with biotinylated DNA sequences specific for the NRP binding site.

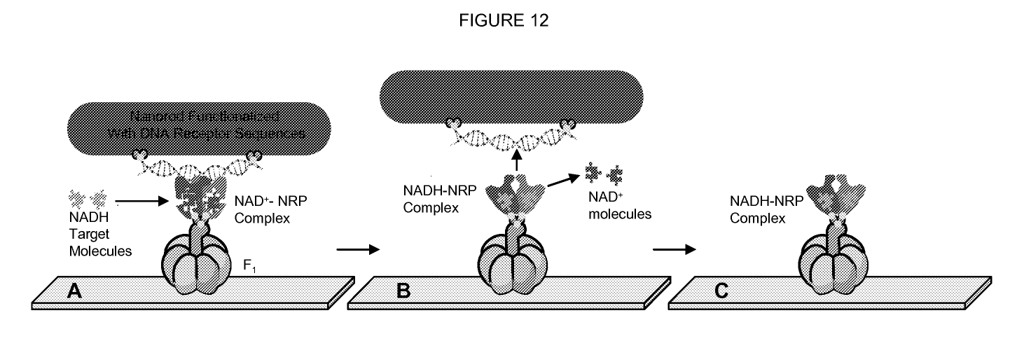

Since during purification, NRP purifies with NADH bound, this dinucleotide will be removed by acidic ammonium sulfate precipitation. Removal of the ammonium sulfate in the presence of NAD⁺ then allows NRP to dimerize with bound NAD⁺. To detect NADH, biotinylated NRP containing bound NAD⁺ will be added to the avidinated F₁-ATPase that had been immobilized on the microscope slide (FIG. 12A-B), and assembly will be completed by addition of gold nanorods functionalized with DNA specific for NRP (FIG. 12C). Detection of NADH will be made by a decrease in the number of nanorods bound to the microscope slide as the binding of the target causes a conformational change that induces the dissociation of the DNA. Since either NAD⁺ or NADH is always bound to NRP, the detection system is capable of determining the ratio between the two.

NRP can also be used in an NAD⁺ detection system. Under these conditions, the slides will be prepared in advance with NADH such that the DNA-functionalized nanorods will not bind. Not to be bound by any theory, we expect that NRP will behave like a transcriptional activating protein to enable assembly with the DNA-coated nanorods upon addition of NAD⁺.

### Example 7

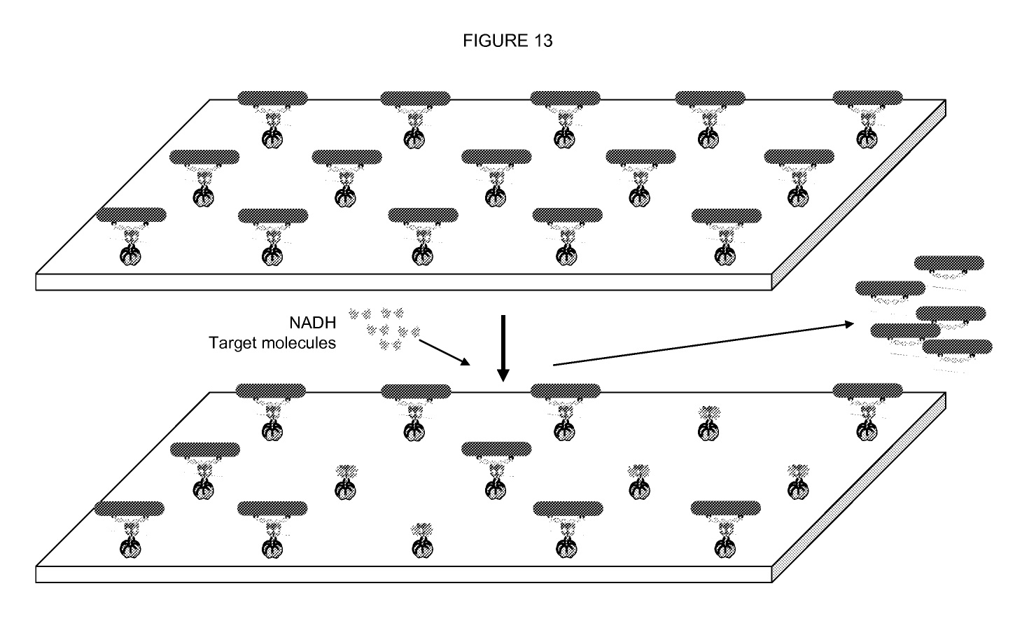

Some embodiments of the present invention provide ordered arrays of nanodevices for metabolite detection containing transcriptional regulator proteins. In some embodiments of the repressor protein-dependent nanodevices, NADH is detected by disassembly of nanodevices as depicted in FIG. 13. Thus, the number of nanorods released will be more easily identified as missing spots of light that interrupt the continuity of the grid. In other embodiments, ordered arrays can be used for detection using the activator-dependent nanodevice system for cAMP detection.

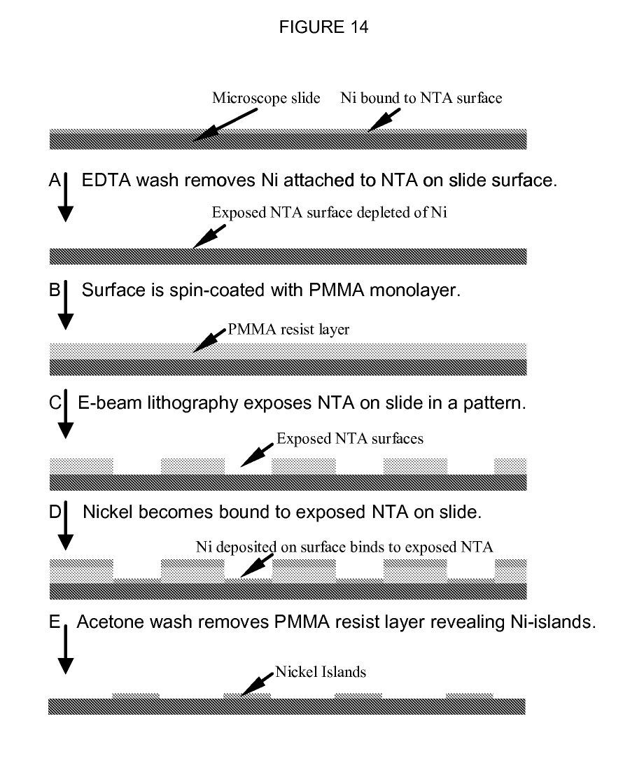

An ordered array will be fabricated by an array of exposed Ni-NTA islands on the surface of the microscope slides, each of which is capable of binding a single F₁ molecule. The fabrication process can be, for example, electron beam lithography as depicted in FIG. 14. First, a clean Ni-NTA coated microscope slide will be flushed with 100 mM EDTA to remove the nickel and washed thoroughly. The silano-NTA remains embedded on the glass surface. The surface is then spin-coated with a monolayer of poly-(methyl methacrylate) (PMMA) resist (˜40 nm). The PMMS resist layer will then be exposed to an E-beam using a predesigned pattern. After subsequent O₂ plasma treatment, nickel sulfate will be added, which will bind to the islands NTA exposed by the E-beam. The O₂ plasma treatment step cleans the surface to facilitate strong adhesion of the nickel. Finally, the surface will be washed with acetone to remove the PMMA. In this manner the surface of the slide will contain and ordered array of nickel islands to which his-tagged-F₁ will bind.

Spacing between nickel islands is far apart enough to prevent one gold nanorod to bridge between two F₁ molecules on adjacent islands, for example about 300 nm. It also provides enough distance so that the rotation of any one nanorod is not affected by the rotation of a nanorod on a neighboring island.

The ordered arrays preferably have one F₁ molecule to every island of Ni-NTA. The nickel islands may have a diameter suitable to provide a base for at least one F₁ molecule to bind.

1. A method for detecting at least one target small molecule metabolite comprising an anchoring part, a bridging part, and a signal producing part wherein:
(a) the anchoring part comprises a molecular motor, wherein the molecular motor is a biological or synthetic molecule capable of induced translational or rotational movements that are capable of being detected;
(b) the bridging part comprises at least one biological or synthetic component that binds to or otherwise responds to the presence of a small metabolite to be detected, wherein said bridging part is linked to said anchoring part through a covalent or high affinity binding interaction;
(c) the signal producing part that comprises at least one of an electromagnetic detection probe that has been functionalized with moieties that specifically bind to or dissociate from said bridging component in the presence of said metabolite to be detected.

2. A method of nanodetection of a target molecule in a sample comprising:
(a) binding each of a plurality of molecular motors to a selected bridging part, wherein said molecular motor is a biological or synthetic molecule capable of detectable translational or rotational movements that are induced and said bridging part is at least one biological or synthetic component that binds to or otherwise responds to the presence of a small metabolite to be detected;
(b) attaching the plurality of molecular motors to a solid support after assembly of said molecular motors with said bridging part;
(c) binding an electromagnetic detection probe specific for the bridging part to the immobilized bridging device wherein said bridging part has a high affinity for said probe in the absence of a target metabolite; and wherein binding of a target metabolite to the bridging part induces a decrease in the affinity of the probe for said bridging part
(d) applying a sample suspected of containing the target metabolite specific for the bridging part;
(e) inducing translational or rotational movement of the at least one molecular motor coupled to the solid support; and
(f) microscopically detecting translational or rotational movement of the at least one molecular motor coupled to the solid support wherein presence of translational or rotational movement in the presence of the sample as indicated by a change in electromagnetic properties of the probe is indicative of lack of target metabolite in the sample and absence of translational or rotational movement as indicated by a lack of change in electromagnetic properties of the probe in the presence of the sample is indicative of presence of the target metabolite in the sample.

3. A method of nanodetection of a target molecule in a sample comprising:
(a) binding each of a plurality of molecular motors to a selected bridging part; wherein said molecular motor is a biological or synthetic molecule capable of detectable translational or rotational movements that are induced and said bridging part is at least one biological or synthetic component that binds to or otherwise responds to the presence of a small metabolite to be detected;
(b) attaching the plurality of molecular motors to a solid support after assembly of said molecular motors with said bridging part;
(c) applying a sample suspected of containing the target metabolite specific to the bridging component in the absence of said electromagnetic probe;
(d) binding of an electromagnetic probe specific for the bridging part wherein said bridging part has a high affinity for the probe when target metabolite specific to said bridging part is bound to said bridging part;
(e) inducing translational or rotational movement of the at least one molecular motor coupled to the solid support; and
(f) microscopically detecting translational or rotational movement of the at least one molecular motor coupled to the solid support by monitoring changes in electromagnetic properties of the probe,
wherein an increased affinity of said probe for the bridging part as indicated by an increase in translational or rotational movement in the presence of the sample indicates the presence of target metabolite specific for that bridging part in the sample, and a decreased affinity of said probe for the bridging part as indicated by a decrease in translational or rotational movement in the presence of the sample indicates the absence of target metabolite specific for that bridging part in the sample.

4. A method of nanodetection of a target molecule in a sample comprising:
(a) binding each of a plurality of molecular motors to a solid support wherein said molecular motor is a biological or synthetic molecule capable of detectable translational or rotational movements that are induced;
(b) linking to each of the plurality of molecular motors a bridging part that comprises at least one biological or synthetic component that binds to or otherwise responds to the presence of a small metabolite to be detected;
(c) binding an electromagnetic detection probe specific for the bridging part to the immobilized bridging device wherein said bridging part has a high affinity for said probe in the absence of a target metabolite, and wherein binding of a target metabolite to the bridging part induces a decrease in the binding affinity of the probe for said bridging part;
(d) applying a sample suspected of containing the target molecule or metabolite specific for the bridging part;
(e) inducing translational or rotational movement of the at least one molecular motor coupled to the solid support; and
(f) microscopically detecting translational or rotational movement of the at least one molecular motor coupled to the solid support wherein presence of translational or rotational movement in the presence of the sample as indicated by a change in electromagnetic properties of the probe is indicative of lack of target metabolite in the sample and absence of translational or rotational movement as indicated by lack of change of electromagnetic properties of the probe is indicative of presence of metabolite in the sample.

5. A method of nanodetection of a target molecule in a sample comprising:
(a) binding each of a plurality of molecular motors to a solid support wherein said molecular motor is a biological or synthetic molecule capable of detectable translational or rotational movements that are induced;
(b) linking to each of the plurality of molecular motors a bridging part that comprises at least one biological or synthetic component that binds to or otherwise responds to the presence of a small metabolite to be detected;
(c) applying a sample suspected of containing the target metabolite specific to the bridging component in the absence of said electromagnetic probe;
(d) binding of an electromagnetic probe specific for the bridging part wherein said bridging part has a high affinity for the probe when target molecule specific to said bridging part is bound to said bridging part;
(e) inducing translational or rotational movement of the at least one molecular motor coupled to the solid support; and
(f) microscopically detecting translational or rotational movement of the at least one molecular motor coupled to the solid support by monitoring changes in electromagnetic properties of the probe,
wherein an increased affinity of said probe for the bridging part as indicated by an increase in translational or rotational movement in the presence of the sample indicates the presence of target metabolite specific for that bridging part in the sample, and a decreased affinity of said probe for the bridging part as indicated by a decrease in translational or rotational movement in the presence of the sample indicates the absence of target metabolite specific for that bridging part in the sample.

6. The method of any of claims 2 through 5 wherein step (c) is performed prior to, after or concurrently with step (d).

7. The method of any of claims 2 through 6, wherein said bridging part is a transcriptional regulator protein that comprises a binding site for a specific DNA sequence and a binding site for said small molecule metabolite and said signal producing part comprises specific DNA sequences known to bind said transcriptional regulator.

8. The method of claim 7, wherein the transcriptional regulator protein is a transcriptional activator protein wherein assembly of said device occurs upon binding of said specific DNA sequences to said transcriptional activator protein which occurs only in the presence of binding of said small molecule metabolite to said transcriptional regulator protein.

9. The method of claim 7, wherein the transcriptional regulator protein is a transcriptional repressor protein wherein assembly of said device occurs upon binding of said specific DNA sequences bound to said transcriptional repressor protein and said electromagnetic detection probe dissociate from said protein in the presence of said small molecule metabolite.

10. The method of any of claims 2 through 6, wherein said bridging part is a signal transduction protein that comprises a binding site for said small molecule metabolite wherein said signal transduction protein changes conformation upon binding to said small molecule metabolite, and said signal producing part comprises an antibody that detects the activated conformation of said signal transduction protein.

11. The method of any of claims 2 through 6, wherein said bridging part is a DNA proofing protein that recognizes modified bases in a DNA sequence, and said signal producing part comprises a DNA sequence that is complementary to the DNA sequence to be detected, wherein assembly of said device occurs upon DNA base-pairing of said DNA to be detected with the DNA sequence affixed to said electromagnetic detection probe.

12. The method of any of claims 2-11, wherein said electromagnetic reporter comprises at least one of an optical, magnetic and thermal particle.

13. The method of claim 12, wherein said electromagnetic reporter comprising an optical reporter comprising at least one of a fluorescent bead and an optical scattering particle.

14. The method of any of claims 2-11 wherein said electromagnetic reporter is a colloidal particle from the elemental group of metals.

15. A nanodetection device comprising an anchoring part, a bridging part and a signal producing part, wherein:
a) the anchoring part comprises a F1-ATPase molecule modified via site directed mutagenesis so as to comprise a his-tag on the N-terminus of an F1-α or F1-β subunit and a cysteine on the F1-γ subunit wherein a plurality of said F1-ATPase molecules are affixed to the surface of a microscope slide such that each F1-ATPase molecule is oriented with the F1-γ subunit away from the surface of said microscope slide, wherein said cysteine on the F1-γ subunit is biotinylated;
b) the bridging part comprises a protein that binds to or otherwise responds to the presence of a small molecule metabolite to be detected, wherein said protein in said bridging part is biotinylated and linked to said anchoring part through a biotin-avidin-biotin linkage with said biotinylated F1-γ subunit of said anchoring part; and
c) the signal producing part comprises an electromagnetic detection probe that has been functionalized with moieties that specifically bind to said protein of said bridging part in the presence of said metabolite to be detected.

16. The nanodetection device of claim 15, wherein said protein in said bridging part is a transcriptional regulator protein that comprises a binding site for a specific DNA sequence and a binding site for said small molecule metabolite and said signal producing part comprises specific DNA sequences known to bind said transcriptional regulator.

17. The nanodetection device of claim 16, wherein the transcriptional regulator protein is a transcriptional activator protein wherein assembly of said device occurs upon binding of said specific DNA sequences to said transcriptional activator protein which occurs only in the presence of binding of said small molecule metabolite to said transcriptional regulator protein.

18. The nanodetection device of claim 16, wherein the transcriptional regulator protein is a transcriptional repressor protein wherein assembly of said device occurs upon binding of said specific DNA sequences bound to said transcriptional repressor protein and said electromagnetic detection probe dissociate from said protein in the presence of said small molecule metabolite.

19. The nanodetection device of claim 15, wherein said protein in said bridging part is a signal transduction protein that comprises a binding site for said small molecule metabolite wherein said signal transduction protein changes conformation upon binding to said small molecule metabolite, and said signal producing part comprises an antibody that detects the activated conformation of said signal transduction protein.

20. The nanodetection device of claim 15, wherein said protein in said bridging part is a DNA proofing protein that recognizes modified bases in a DNA sequence, and said signal producing part comprises a DNA sequence that is complementary to the DNA sequence to be detected, wherein assembly of said device occurs upon DNA base-pairing of said DNA to be detected with the DNA sequence affixed to said electromagnetic detection probe.

21. The nanodetection device of any of claims 15 through 20, wherein said electromagnetic reporter comprises at least one of an optical, magnetic and thermal particle.

22. The nanodetection device of claim 21, wherein said electromagnetic reporter comprising an optical reporter comprising at least one of a fluorescent bead and an optical scattering particle.

23. The nanodetection device of any of claims 15 through 20 wherein said electromagnetic reporter is a colloidal particle from the elemental group of metals.

24. A method for detecting whether a molecule binds an activator of a transcriptional regulator protein comprising:
a) preparing a nanodetection device according to claim 15,
b) contacting said device with a target small molecule metabolite;
c) adding ATP to said device under conditions to allow activity of F1-ATPase to rotate the F1-γ subunit; and
d) comparing the signal produced from the rotating electromagnetic detection probes bound to the microscope slide in the presence of said target metabolite with the signal produced by probes in the absence of said target metabolite wherein an increase in the signal in the presence of said metabolite indicates that said metabolite is bound to said transcriptional regulator protein.

25. A method for detection of a molecule that binds a repressor protein
a) preparing a nanodetection device according to claim 18,
b) contacting said device with a target small molecule metabolite;
c) adding ATP to said device under conditions to allow activity of F1-ATPase to rotate the F1-γ subunit; and
d) comparing the signal produced from the rotating electromagnetic detection probes bound to the microscope slide in the presence of said target metabolite with the signal produced by probes in the absence of said target metabolite wherein a decrease in the signal in the presence of said metabolite indicates that said metabolite is bound to said repressor protein.

26. A method of detecting binding of a molecule to a signal transduction protein comprising:
a) preparing a nanodetection device of claim 19;
b) contacting said device with a target small molecule metabolite;
c) adding ATP to said device under conditions to allow activity of F1-ATPase to rotate the F1-γ subunit; and
d) comparing the signal produced from the rotating electromagnetic detection probes bound to the microscope slide in the presence of said target metabolite with the signal produced by probes in the absence of said target metabolite wherein an increase in the signal in the presence of said metabolite indicates that said metabolite is bound to said signal transduction protein.

27. A method of proofing DNA comprising:
a) preparing a nanodetection device of claim 20;
b) contacting said device with a DNA sequence to be proofed;
c) adding ATP to said device under conditions to allow activity of F1-ATPase to rotate the F1-γ subunit; and
d) determining the presence of a modified DNA in the DNA sequence to be proofed by determining the signal produced from the rotating electromagnetic detection probes bound to the microscope slide in the presence of said DNA to be proofed wherein a signal is produced when there is DNA base-pairing with a DNA sequence affixed to said electromagnetic detection probe.

28. The method of any of claim 24, 25, 26 or 27 wherein step (d) comprises detecting the signal using visual detection by dark field microscopy.

29. The method of any of claim 24, 25, 26 or 27 wherein step (d) comprises determining an oscillation of intensity of light at one or more wavelengths from the detection probe.

30. A kit comprising
(a) a protein that specifically binds the small molecule metabolite of interest, wherein the protein is biotinylated in a manner that does not interfere with the binding of the small molecule metabolite of interest;
(b) F1-ATPase molecule wherein the F1-ATPase molecule has a his-tag to facilitate attachment of the F1-ATPase to a solid support and further wherein the F1-ATPase is biotinylated; and
(c) gold nanorods bound to molecules that recognize the protein in (a) when the protein is bound to a small molecule metabolite of interest.

31. The kit of claim 30 further comprising a solid support.

32. The kit of claim 30 further comprising avidin.
